(19) United States
(12) Patent Application Publication
(10) Pub. No.: US 2026/0356938 A1
(43) Pub. Date: Nov. 12, 2026

(54) REBALANCING THE IMMUNE SYSTEM THROUGH DEPLETION OF MYELOID-BIASED HEMATOPOIETIC STEM CELLS

(71) Applicants: The Board of Trustees of the Leland Stanford Junior University, Stanford, CA (US); The United States of America, as represented by the Secretary, Department of Health and Human, Bethesda, MD (US)

(72) Inventors: Jason B. Ross, Cupertino, CA (US); Lara Myers, Redwood City, CA (US); Joseph Noh, Redwood City, CA (US); Kim J. Hasenkrug, Victor, MT (US); Irving L. Weissman, Stanford, CA (US)

(21) Appl. No.: 19/121,769

(22) PCT Filed: Oct. 30, 2023
(86) PCT No.: PCT/US2023/036280

§ 371 Date: Apr. 16, 2025

Related U.S. Application Data
(60) Provisional application No. 63/422,765, filed on Nov. 4, 2022.

(51) Int. Cl. C07K 16/28; A61K 39/00; A61P 37/06

(52) U.S. Cl. CPC C07K 16/2803; A61P 37/06; C07K 16/28; C07K 16/2854; A61K 2039/507; C07K 2317/76

(57) ABSTRACT

The present disclosure provides a clinically applicable method to re-balance production of hematopoietic cells in a mammal, including without limitation an aged mammal, by selective depletion of myeloid-biased hematopoietic stem cells (my-HSC). This selective depletion provides for increased production of lymphoid cells relative to myeloid cells.

### CROSS REFERENCE TO OTHER APPLICATIONS

This application claims the benefit of PCT Application No. PCT/US2023/036280, filed Oct. 30, 2023, which claims benefit of U.S. Provisional Patent Application No. 63/422,765, filed Nov. 4, 2022, the contents of which applications are hereby incorporated by reference in their entirety.

### GOVERNMENT SUPPORT RESEARCH

This invention was made with Government support under contracts AI143889, CA220434, and DK115600 awarded by the National Institutes of Health. The Government has certain rights in the invention.

### BACKGROUND

Hematopoietic stem cells (HSC) can exist in at least two distinct subsets-one subset that provides balanced production of myeloid and lymphoid cells, referred to herein as “bal-HSC”, and another that is biased toward production of myeloid lineage cells, referred to herein as “my-HSC”. Aging is associated with a transition to mainly myeloid biased HSC. There are at least four critical problems associated with this transition. First, myeloid biased HSC reduce the number of naive lymphoid cells in aged individuals, leading to poor T and B cell responses to new pathogens, including microbes such as SARS-COV-2, influenza, HIV, etc. and vaccine responses. Second, myeloid biased HSC contribute to a chronic inflammatory milieu in the aged (known as inflammaging) that is associated with numerous age-related pathologies. Third, myeloid biased HSC can transform to cause human hematopoietic diseases such as MPN (myeloproliferative neoplasms), MDS (myelodysplastic syndrome), chronic myelogenous leukemia (CML), acute myelogenous leukemia (AML), and clonal hematopoiesis of indeterminate potential (CHIP). Fourth, loss of adaptive T and B cell responses in the aged is associated with an increased incidence of cancer.

Animals, including humans, respond well to the microbes in their local geography, first by eliciting an innate immune response predominated by cells of the myeloid lineage (such as macrophages, neutrophils and granulocytes), and secondly by eliciting a much more specific adaptive response by lymphocytes (B cells and T cells). In youth, each of the millions of B cells and T cells expresses a distinct receptor capable of recognizing a specific antigen from a pathogen. Upon pathogen encounter, those cells with specificity expand into both effector cells to contain the pathogen, and into long-lived memory cells that can respond much faster and more potently if the pathogen is re-encountered. HSC give rise to both the common myeloid progenitors (CMP) necessary for innate immunity and the common lymphocyte progenitors (CLP) necessary for adaptive immunity. In young individuals the output of both lineages is balanced. As individuals age, the myeloid biased HSC start to dominate, and the lymphoid compartment becomes increasingly comprised of memory cells from previous encounters, rather than naive cells able to respond to novel microbial pathogens. The advent of modern transportation has thus brought novel pathogens (such as SARS-COV-2) from the far reaches of the world to infect elderly individuals with no immunological memory and little capacity to respond to novel pathogens.

Another critical problem with aged immune systems is that the myeloid biased HSC are pro-inflammatory, producing or eliciting inflammatory cytokines such as TNFα, IL1, IL6, etc. As made evident during the recent pandemic, unbalanced inflammatory responses in the elderly cause much more morbidity and mortality due to inflamed and fibrotic lungs. Thus, the predominance of myeloid biased HSCs in the elderly is a two-edged sword in the battle with novel pathogens, resulting not only in a poor adaptive immune response, but also in a detrimental inflammatory response.

In addition to problems with infectious diseases and hematopoietic diseases, almost all cancers have an increased incidence as humans age. While this is in part due to the accumulation of driver mutations in precancerous clones over long periods of time, the diminution of adaptive immunity and the confounding presence of a more inflammatory milieu in the aged likely contributes to an inability to recognize and eliminate newly arising cancers. Thus, rejuvenation of the immune system with a more balanced HSC subset can restore the surveillance system required for recognition not only of pathogenic challenges, but also transformed and partially transformed cells that cause hematopoietic diseases and other cancers.

To address these pathologies, therapeutic methods are disclosed herein to restore HSCs to a balanced production of lymphoid and myeloid lineage cells.

### SUMMARY OF THE INVENTION

Compositions and methods are provided for rebalancing the immune system of a mammalian subject, including without limitation an aged mammal, by selective depletion of myeloid-biased hematopoietic stem cells (my-HSC). The effect of my-HSC depletion is to shift production of differentiated immune cells from the pool of balanced hematopoietic stem cells (bal-HSC). Compared to my-HSC, bal-HSC generate greater numbers of lymphoid progenitor cells relative to myeloid progenitor cells. The result of this selective depletion can be a relative enhancement of circulating naïve lymphocyte populations, and decreased myeloid cell populations and exhausted T cell populations. The rebalanced immune system has an improved capacity to respond to novel infections, including vaccinations, and has reduced inflammaging properties. Conditions that can be treated with the methods include, for example, clonal hematopoiesis of indeterminate potential (CHIP), myeloproliferative neoplasms (MPN), myelodysplastic syndrome (MDS), acute myeloid leukemia (AML), including pre-malignant AML, atherosclerosis, inflammatory and fibrotic conditions, pathogenic infections, e.g. influenza, Covid-19, etc., inadequate response to vaccination, prevention or treatment of liquid and solid cancers, immune recovery after cytotoxic agents, and the like.

Human HSC can be phenotyped by their expression of cell surface markers, and on the basis of this expression that can be categorized as my-HSC or bal-HSC. For example, all human HSC are positive for expression of CD34, CD90, and CD117. The disclosure herein identifies cell surface markers that are differentially expressed on human and mouse my-HSC relative to bal-HSC, which markers are used in the selective depletion of my-HSC. The markers may be referred to herein as “my-HSC selective markers”. In some embodiments, the my-HSC selective markers comprise one or more of CD304, TIE2, ESAM, CD9, CD105, CD166, CD150 (Slamf1), CD61 (Itgb3), CD41 (Itga2b), CD62p, and NEO1. In some embodiments the my-HSC selective markers comprise one or more of CD150 (Slamf1), CD61 (Itgb3), CD41 (Itga2b), CD62p, and NEO1. Human my-HSC selective markers include, for example, CD150, NEO1 and CD62p.

Methods are provided for the in vivo or in vitro selective depletion of my-HSC relative to bal-HSC, for example by selective immunodepletion. The methods comprise contacting a population of cells, e.g. cells in bone marrow, comprising HSC with an effective dose of one or more agents that specifically bind to a my-HSC selective marker, which may be referred to as a my-HSC selective agent. In some embodiments a cocktail of binding agents is used, which bind to a plurality of my-HSC selective markers. In some embodiments the my-HSC selective marker is CD150. In some embodiments the my-HSC selective marker is CD62p. In some embodiments the my-HSC selective marker is NEO1.

In some embodiments, methods of selective immunodepletion comprise administering an effective dose of an agent specific for CD117 in combination with the my-HSC selective agent(s). In some embodiments, methods of selective immunodepletion comprise administering an effective dose of an agent that blocks CD47 interaction with SIRPα, in combination with the my-HSC selective agent(s). In some embodiment, methods of selective immunodepletion comprise administering an effective dose of an agent specific for CD117, and an agent that blocks CD47 interaction with SIRPα, in combination with the my-HSC selective agent(s).

In some embodiments a cocktail of antibodies is administered, comprising an antibody specific for CD47, an antibody specific for a my-HSC marker, for example one or more of anti-CD150, anti-CD62p, anti-NEO1, and an antibody specific for CD117. In some embodiments a cocktail of agents is administered, comprising an antibody specific for SIRPα, an antibody specific for a my-HSC marker, for example one or more of anti-CD150, anti-CD62p, anti-NEO1, and an antibody specific for CD117. In some embodiments a cocktail of agent is administered, comprising a soluble SIRPα polypeptide, an antibody specific for a my-HSC marker, for example one or more of anti-CD150, anti-CD62p, anti-NEO1, and an antibody specific for CD117.

In some embodiments one or all of the agents (my-HSC selective agent, CD117 specific agent, agent that blocks CD47 interaction with SIRPα) is an antibody. In some embodiments the antibody is a humanized monoclonal antibody. An antibody may comprise an Fc region sequence. In some embodiments, a single dose of the antibody is administered in vivo. In some embodiments the dose of antibody is delivered by intravenous infusion. The effective dose of the antibody may be up to about 50 mg/kg, up to about 25 mg/kg, up to about 10 mg/kg; up to about 5 mg/kg; up to about 1 mg/kg; up to about 0.1 mg/kg. In some embodiments an antibody dose is from about 0.1 mg/kg to about 25 mg/kg, from about 0.5 mg/kg to about 15 mg/kg, from about 1 to about 5 mg/kg. The antibody is optionally conjugated to a cytotoxic agent.

In certain embodiments the subject being treated is an aged, or elderly, mammal. The rate of aging is species specific, where a human may be aged at about 50 years; and a rodent at about 2 years. In general terms, a natural progressive decline in body systems starts in early adulthood, but it becomes most evident several decades later. One arbitrary way to define elderly more precisely in humans is to say that it begins at conventional retirement age, around about 60, around about 65 years of age. Another definition sets parameters for aging coincident with the loss of reproductive ability, which is around about age 45, more usually around about 50 in humans, but will, however, vary with the individual.

In some embodiments an individual diagnosed with CHIP, or a myelodysplastic condition, e.g. pre-malignant AML, MDS, and the like is treated with the methods disclosed herein to rebalance the immune system and to shift production of differentiated immune cells to balanced hematopoietic stem cells (bal-HSC).

The method of selective depletion may provide for an enrichment of bal-HSC to my-HSC of at least 1.5-fold, at least 2-fold, at least 3-fold, at least 5-fold, at least 7.5-fold, at least 10-fold, at least 15-fold, or more. After a period of time sufficient for rebalancing, e.g. after about 1 week, after about 2 weeks, after about 3 weeks, the ratio of the number of lymphoid progenitors in bone marrow, e.g. common lymphoid progenitors, to the number of myeloid progenitors, e.g. common myeloid progenitors, may be increased at least 1.5-fold, at least 2-fold, at least 3-fold, at least 5-fold, at least 7.5-fold, at least 10-fold, at least 15-fold, or more. The number of circulating naïve T cells relative to the total circulating lymphocyte population may be increased at least 2-fold, at least 3-fold, at least 5-fold, at least 7.5-fold, at least 10-fold, at least 15-fold, or more. The level of circulating “age-associated B cells” (ABC), and/or exhausted T cells relative to the total circulating lymphocyte population may be decreased at least 1.5-fold, at least 2-fold, at least 3-fold, at least 5-fold, at least 7.5-fold, at least 10-fold, at least 15-fold, or more. The basal circulating level of ‘inflammaging’ markers, e.g. IL-1a, CXCL5, IL1RL1, IL-23, IL-1b, CXCL2, IL-31, IL-5, GM-CSF, may be decreased at least at least 1.5-fold, 2-fold, at least 3-fold, at least 5-fold, at least 7.5-fold, at least 10-fold, at least 15-fold, or more upon treatment with the methods of the invention.

In some embodiments the balance of immune cells, e.g. the relative number of one or more of naïve T cells, exhausted T cells, ABC, myeloid progenitors and lymphoid progenitors is determined before my-HSC-selective depletion. In some embodiments the balance of immune cells, e.g. the relative number of one or more of naïve T cells, exhausted T cells, ABC, myeloid progenitors and lymphoid progenitors is determined before my-HSC-selective depletion, where an improvement in the desired balance of lymphoid to myeloid cells is associated with successful selective depletion.

The method of selective depletion may provide for an improved immune response, e.g. response to viral infection; response to bacterial infection; response to pre-malignant or malignant tumor; response to vaccination; generation of antibodies in response to an immunogen; and the like, relative to the individual's response prior to treatment with the methods of the disclosure. For example and without limitation, an antigen-specific CD8+ T cell response can be increased at least 1.5-fold, at least 2-fold, at least 3-fold, at least 5-fold, at least 7.5-fold, at least 10-fold, at least 15-fold, or more in a rebalanced individual. An antigen-specific antibody response can be increased at least 1.5-fold, at least 2-fold, at least 3-fold, at least 5-fold, at least 7.5-fold, at least 10-fold, at least 15-fold, or more in a rebalanced individual. The severity of infection or tumor burden may be reduced, e.g. a decrease in hospitalization, infected cells, mortality, tumor burden, metastases, cancer relapse and the like.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-1L. Identification of cell-surface antigens on myeloid-biased hematopoietic stem cells. a, Schematic to identify genes encoding candidate myeloid-biased HSC cell-surface antigens (left) and validate their presence on my-HSCs vs. bal-HSCs (right). b, Heatmap depicting expression of candidate markers across independent datasets (left), with ranked mean log2 fold-change (Old HSC vs. Young HSC; My-HSC vs. Bal-HSC) of each candidate (right). Datasets include comparison of Old vs. Young HSCs (a, b, c, d, e, f, g, h) and My-HSCs vs. Bal-HSC (i, j, k). c, Comparison of percent-positive for each candidate marker on my-HSCs vs. bal-HSCs (left); plot of my-HSC fold-enrichment for each marker, calculated as: (% marker positive of my-HSC)/(% marker positive of bal-HSC) (right). d, Plot of (my-HSC)/(bal-HSC) Fold-Change (log2) of RNA expression (y-axis) vs. Cell-surface Protein expression (x-axis) for 12 candidate markers. e, Representative flow-cytometry gating to identify total HSCs (KLS FLT3⁻CD34⁻CD150⁺), myeloid-biased HSCs (my-HSCs; KLS FLT3⁻CD34⁻CD150^{High}), and balanced HSCs (bal-HSCs; KLS FLT3⁻CD34⁻CD150^{Low}) from mouse bone marrow. Panels depict gating after exclusion of dead cells, doublets, and gated on KLS (Lin⁻cKIT⁺Sca1⁺). f, Representative flow-cytometry of percent of bal-HSCs (left) or my-HSCs (right) that are NEO1⁺ or CD62p⁺. g-i, Relative cell-surface expression of CD150 (g), NEO1 (h), and CD62p (i), on total HSC (my-HSC & bal-HSC), my-HSC, bal-HSC, MPPa, MPPb, MPPc, CMP (CMP&GMP), MkP, and CLP populations. Flow-cytometry median fluorescent intensity (MFI) values for antibodies to each marker were obtained for each population and normalized from 0-1 based on the lowest to highest expression. j-I, Comparison of the percentage of total HSCs (KLS FLT3⁻CD34⁻CD150⁺) that are my-HSCs (e.g., CD150High) (j), NEO1+(k), or CD62p⁺ (I) on the y-axis, vs. mouse age in weeks on the x-axis; n=21 mice (j-k); n=10 mice (I). Mouse ages were approximately: 4-6 months (c-i), or 6-12 months (j-l). For (c-l), bone-marrow was cKIT-enriched prior to FACS analysis. p-values and R values calculated with one-tailed Pearson correlation coefficient (j-l). FC, fold-change; RNA-seq, RNA-sequencing.

FIGS. 2A-2K. Antibody-mediated depletion of myeloid-biased hematopoietic stem cells in vivo. a, Schematic of strategy to restore balanced lineage output from HSCs by depleting my-HSCs using antibody-conditioning. b, Schematic to deplete my-HSCs by targeting my-HSC specific antigens (CD150, CD62p, or NEO1), in combination with antibodies to CD47 and to cKIT (left), followed by phenotypic analysis (right). c-e, Percentage of total HSCs that are my-HSCs (left) in mice receiving: anti-CD150 (c), anti-CD62p (d), or anti-NEO1 (e), optimized antibody-conditioning protocols, which include anti-CD47 and anti-cKIT. Representative flow-cytometry of total HSCs (KLS FLT3⁻CD34⁻CD150⁺) that are my-HSC (CD150^{High}) or bal-HSC (CD150^{Low}) for each protocol (right); n=3 mice (c, e); n=4 mice (d). f-h, Enrichment (blue) or depletion (red) as percentage of live cells for total HSCs (HSC), my-HSCs (My), bal-HSCs (Bal), MPPs, and lymphoid (CLP) and myeloid progenitors (CMP&GMP, MkP) in mice receiving: anti-CD150 (f), anti-CD62p (g), or anti-NEO1 (h), optimized antibody-conditioning protocols (e.g., +anti-CD47 and +anti-cKIT). Values are relative to the mean of control mice and log2-transformed. Values for treated mice are in closed filled circles; values for control mice are in open unfilled circles; n=3 mice (f, h); n=4 mice (g). i-k, Total HSCs (KLS FLT3⁻CD34⁻CD150⁺) were FACS-sorted from aged control mice (Aged, A) or aged mice with my-HSC depletion (Aged+Conditioning, A+C) and underwent bulk RNA-sequencing. My-HSC depletion was performed with anti-NEO1+anti-CD62p+anti-cKIT/CD47 and cells were collected at day 9 post-treatment. i, FPKM of top 200 differentially expressed genes between A HSCs vs. A+C HSCs ranked by p-value. j-k, GSEA applied to differentially expressed genes of A HSCs vs. A+C HSCs, ranked by test statistic as input, using gene-signatures obtained from Young HSC vs. Old HSC (j, i, ii, iii, iv, v, vi, vii, viii) or bal-HSC vs. my-HSC. (k, i, ii, iii) datasets; n=3 mice (A), n=3 mice (A+C). Mouse ages were approximately: 5-9 months (c-h), or 11 months (i-k). For (c-h), bone-marrow was cKIT-enriched prior to FACS analysis. P-values were obtained by unpaired parametric one-tailed t-test (c-h), or by GSEA with false discovery rate (FDR) adjustment (j-k). FC, Fold-Change; Rel., relative. A, Aged. A+C, Aged+Conditioning. GSEA, Gene Set Enrichment Analysis. FPKM, Fragments Per Kilobase of transcript per Million mapped reads.

FIGS. 3A-3J. Depletion of my-HSCs in aged mice restores features of a youthful immune system. a, Schematic of time-course experiment to determine the impact of antibody-conditioning on aged mice. Young-adult mice (Y) were compared to aged mice (A), with or without antibody conditioning with anti-NEO1 optimized protocol (A+C), at approximately 1-, 8-, or 16-weeks post-treatment. b, Relative frequency as percentage of live cells for my-HSCs and CLPs in the bone-marrow of aged mice receiving antibody-conditioning (A+C), compared to age-matched controls (A), approximately 1-, 8-, or 16-weeks post-treatment; n=6 mice (A, A+C) at 1-week; n=4 mice (A), n=9 mice (A+C) at 8-weeks; n=4 mice (A, A+C) at 16-weeks. Values are relative to the mean value for aged control mice at each time-point and log2-transformed. Values for aged mice receiving antibody-conditioning are in closed filled circles; values for control aged mice are in open unfilled circles. c, Frequency of my-HSCs as a percentage of live cells in young-adult (Y), aged (A), or aged+conditioning mice (A+C), with representative flow-cytometry (right); n=5 mice (Y), n=6 mice (A, A+C). d, Frequency of CLPs as a percentage of live cells in young-adult (Y), aged (A), or aged+conditioning mice (A+C), with representative flow-cytometry (right); n=4 mice (Y, A, A+C). e, Naïve T cells (CD44⁻CD62L⁺) as a percentage of total CD4 & CD8 T cells, in young-adult (Y), aged (A), or aged+conditioning mice (A+C), with representative flow-cytometry (right); n=6 mice (Y, A), n=5 mice (A+C). f, Mature B cells (IgM+IgD+) as percentage of total B cells (CD19⁺B220⁺), in young-adult (Y), aged (A), or aged+conditioning mice (A+C), with representative flow-cytometry (right); n=6 mice (Y, A), n=5 mice (A+C). g, CD4 T cell exhaustion ratios (percent PD1⁺CD62L⁻)/(percent PD1⁻CD62L⁺), in young-adult (Y), aged (A), or aged+conditioning mice (A+C), with representative flow-cytometry (right); n=6 mice (Y, A), n=5 mice (A+C). h, Aged B Cells (ABCs) (CD21/CD35 CD23) as percentage of total mature B cells (CD19⁺IgM+CD93⁻CD43⁻), in young-adult (Y), aged (A), or aged+conditioning mice (A+C), with representative flow-cytometry (right); n=6 mice (Y, A), n=5 mice (A+C). i, Estimated plasma concentration of IL-1α, in young-adult (Y), aged (A), or aged+conditioning mice (A+C); n=6 mice (Y, A), n=5 mice (A+C). j, Relative plasma abundance of the top 17% of proteins, ranked by statistical significance, increased in aged (A) vs. aged+conditioning (A+C) mice. Values are relative to the mean value for young mice (Y) and log2-transformed. Gray squares depict samples with no data or removed statistical outliers. Statistical significance is represented as −log₁₀p of comparison between (A) vs. (A+C) mice; n=6 mice (Y, A), n=5 mice (A+C). For a-j, data was obtained at approximately 1-week (b, c), 8-weeks (b, g-j), or 16-weeks (b, d) after antibody-conditioning. Mouse ages are at time of analysis: Y, young-adult (3-6 months) mice; A, aged (18-24 months) mice; A+C, aged (18-24 months) mice receiving antibody-conditioning. For (b-d), bone-marrow was cKIT-enriched prior to analysis. p-values were obtained by unpaired parametric one-tailed t-test (b), by ordinary one-way ANOVA followed by one-tailed Dunnett's multiple comparison test using Aged as control (c-d, f-i), by Brown-Forsythe and Welch ANOVA followed by Dunnett's T3 multiple comparisons test using Aged as control (e), or by one-way ANOVA followed by Holm multiple comparisons test (j). n.d., not determined.

FIGS. 4A-4E. Antibody-conditioning enhances functional immunity to infection in aged mice. a, Frequency of my-HSCs as percentage of live cells in young-adult (3-5 months) mice (Y), aged (21 months) mice (A), or aged+conditioning (21 months) mice (A+C) approximately 10-weeks after anti-NEO1^{v2} conditioning protocol, with representative flow-cytometry (right). Aged and aged+conditioning mice were vaccinated at 8-weeks after anti-NEO1; n=2 mice (Y), n=7 mice (A), n=10 mice (A+C). b, Percentage of CD8+ T cells in the spleen that are FV antigen-specific (Dextramer+CD44⁺) in aged-matched (20-26 months) mice without (A), or with antibody-conditioning (A+C), 10-14 days after intravenous (i.v.) vaccination with live-attenuated virus (left). My-HSC depletion was conducted 2 months prior to vaccination with anti-NEO1^{v2} conditioning protocol. Representative FACS plots of spleens from vaccinated mice (right), gated on CD8⁺ T cells; n=13 mice (A, vaccinated), n=15 mice (A+C, vaccinated), pooled from two independent experiments. c, Spleen weight for young-adult (Y), aged (A), or aged+conditioning mice (A+C) that were (i) Naïve, (ii) Infected with Friend Virus (FV), or (iii) Vaccinated & Infected with FV; n=9 mice (Y, naïve), n=13 mice (Y, FV infected), n=13 mice (Y, vaccinated & FV infected), n=6 mice (A, naïve), n=10 mice (A, FV infected), n=8 mice (A, vaccinated & FV infected), n=5 mice (A+C, naïve), n=9 mice (A+C, vaccinated & FV infected). Units are in mg and log10-transformed. Graph bars depict median. c, Infectious virus levels in young-adult (Y), aged (A), or aged+conditioning mice (A+C) that were (i) Infected with FV, or (ii) Vaccinated & Infected with FV; n=13 mice (Y, FV infected), n=13 mice (Y, vaccinated & FV infected), n=10 mice (A, FV infected), n=8 mice (A, vaccinated & FV infected), n=9 mice (A+C, vaccinated & FV infected). Data is log10-transformed. Graph bars depict median. e, Percent of CD8+ T cells in the spleen that are FV antigen-specific (Dextramer⁺CD44⁺) in vaccinated aged mice without (A), or with antibody conditioning (A+C), 14 days after infection with FV (left). Representative FACS plots of spleens from vaccinated mice (right), gated on CD8⁺ T cells; n=8 mice (A, vaccinated & FV infected), n=9 mice (A+C, vaccinated & FV infected). For c-e, mouse ages are at time of analysis: Y, young-adult (3-6 months) mice; A, aged (21-22 months) mice; A+C, aged (21-22 months) mice receiving antibody-conditioning. For c-e, data from experiments using the anti-NEO1^{v1} conditioning protocol (open circles) or the anti-NEO1^{v2} conditioning protocol (closed circles) were combined. For (a), bone-marrow was cKIT-enriched prior to analysis. p-values were obtained by ordinary one-way ANOVA followed by one-tailed Dunnett's multiple comparisons test using Aged as control (a), by one-tailed unpaired parametric t-test (b), by ordinary one-way ANOVA followed by Tukey's multiple comparisons test (c-d), or by one-tailed unpaired parametric t-test with Welch's correction (e). Y, young-adult mice; A, aged mice; A+C, aged mice receiving antibody-conditioning; NEO1^{v2} protocol is NEO1^{v1} protocol (anti-NEO1+anti-CD47+anti-cKIT)+mouse-IgG2a secondary antibody; LOD, limit of detection; n.s., not significant.

FIGS. 5A-5J. Mouse myeloid-biased HSC markers are enriched in aged human HSCs. a, Heatmap depicting RNA expression of candidate human my-HSC antigens in independent datasets of human Old vs. Young HSCs (a, b, c, d). b, Relative RNA expression of CD62p (Selp), CD41 (Itga2b), CD150 (Slamf1), and NEO1 (Neo1) in human HSCs isolated from young (ages 20-31), middle & old (ages 42-85) donors. c-e, Correlation of relative RNA expression of CD62p (c), CD41 (d), and CD150 (e) in human HSCs compared to donor age. f, Representative flow-cytometry of CD34⁺-enriched donor bone-marrow to identify human HSCs (Lin⁻CD34⁺CD38⁻CD45RA⁻CD90⁺). For b-e, values are relative to mean of young samples. g, Representative flow-cytometry staining of HSCs with anti-CD62p antibody (red) compared to fluorescence-minus-one (FMO) control (black); n=3 independent donors. h-i, Histograms for flow-cytometry staining of HSCs with antibodies to CD304, CD150, TIE2, CD62p, ESAM, CD9, CD47, CD105, CD166; black line represents FMO control (h), with percent of HSCs positive for each marker (i). j, Model to rejuvenate aged immune systems by depleting myeloid-biased hematopoietic stem cells. p-values were obtained by unpaired parametric one-tailed t-test (b), or p-values and R values calculated with one-tailed Pearson correlation coefficient (c-e). FMO, fluorescence-minus-one.

FIGS. 6A-6O. Expression of candidate my-HSC markers in hematopoietic progenitors, mature cells, and non-hematopoietic tissues. a-l, Expression of my-HSC candidate markers, Slamf1 (CD150) (a), Neo1 (NEO1) (b), Itga2b (CD41) (c), Selp (CD62p) (d), Cd38 (CD38) (e), Itgb3 (CD61) (f), Itgav (CD51) (g), Procr (CD201) (h), Tie2 (i), Esam (j), Eng (CD105) (k), Cd9 (CD9) (l), in hematopoietic stem and progenitor cells (HSPCs) in normal mouse bone marrow (top panels), and in young versus old bone marrow (bottom panels). Data from a-I obtained from Gene Expression Commons. m, Heatmap of relative RNA expression for CD150 (Slamf1), NEO1 (Neo1), CD62p(Selp), CD41(Itga2b), CD38 (Cd38), CD51 (Itgav), and CD6 1 (Itgb 3) in HSCs, MPPs, Progenitors, Myeloid, and Lymphoid cells. Processed data for 23 cell types were obtained directly from Gulati. Fold-enrichment=[(average percentile of HSCs)/(average percentile of all other cell types)+100], as described in this publication. n-o, RNA expression of CD150 (Slamf1), NEO1 (Neo1), CD62p (Selp), and CD41 (Itga2b) in bulk mouse tissues from two independent datasets: Tabula Muris (n, GSE132040) or Kadoki (o, GSE87633). For n-o, Values are z-score normalized for each gene across all tissues.

FIGS. 7A-7K. Gating strategy for total HSCs, my-HSCs, bal-HSCs, and HPCs. a-b, Representative flow-cytometry gating of mouse bone-marrow to identify total HSC (Lin⁻cKIT⁺Sca1⁺FLT3⁻CD34⁻CD150⁺), myeloid-biased HSC (my-HSC; Lin⁻cKIT⁺Sca1⁺FLT3⁻CD34⁻CD150^{High}), balanced HSC (bal-HSC; Lin⁻cKIT⁺Sca1⁺FLT3⁻CD34⁻CD150^{Low}), MPPa (Lin⁻cKIT⁺Sca1⁺FLT3⁻CD34⁺CD150⁺), MPPb (Lin⁻cKIT⁺Sca1⁺FLT3⁻CD34⁺CD150⁻), MPPc (Lin⁻cKIT⁺Sca1⁺FLT3⁺CD34⁺CD150⁻), OPP (Lin⁻cKIT⁺Sca1⁻) , CMP&GMP (Lin⁻cKIT⁺Sca1⁻CD34⁺CD41⁻), MkP (Lin⁻cKIT⁺Sca1⁻CD34⁺CD41⁺), MEP (Lin⁻cKIT⁺Sca1⁻CD34⁻CD41⁺), and CLP (Lin⁻cKIT^{Lo}Sca1^{Lo}IL7Ra+FLT3⁺). Panels depict gating after exclusion of dead cells, doublets, and lineage-positive (CD3⁺, or Ly-6G⁺/C⁺, or CD11b⁺, or CD45R⁺, or Ter-119⁺) cells. Illustration depicting Hematopoietic Stem and Progenitor Cell (HSPC) Tree Analysis (b), with colors for each cell population corresponding to gating scheme in (a). CMP is combined CMP&GMP. Gate to define my-HSC vs. bal-HSC was set as described previously⁷. c-f, Relative expression of CD41 (c), CD38 (d), CD51 (e), CD61 (f), on total HSC, my-HSC, bal-HSC, MPPa, MPPb, MPPc, CMP&GMP, MkP, and CLP. Flow-cytometry median fluorescent intensity (MFI) values for each marker were obtained for each population and normalized from 0-1 based on the lowest to highest expression. g-h, Relative cell-surface levels (g) and percent-positive cells (h) for CD150, NEO1, CD62p, CD41, CD38, CD51, and CD61, on lineage-positive (CD3⁺, or Ly-6G+/C⁺, or CD11b⁺, or CD45R⁺, or Ter-119⁺) high and low cells, total HSCs (my-HSCs+bal-HSCs), and HPCs in the bone-marrow. For cell-surface levels (g), MFI values for each marker were obtained for each population and normalized from 0 -100 based on the lowest to highest expression. i, Comparison of the percentage of total HSCs (KLS FLT3⁻CD34⁻CD150⁺) that are CD41⁺ on the y-axis vs. mouse age in weeks on the x-axis; n=21 mice. j, Comparison of mouse age (x-axis) vs. the frequency of total HSCs (my-HSC+bal-HSC) as a percentage of live cells in the (i) total bone-marrow (left y-axis, red) or (ii) cKIT-enriched bone-marrow (right y-axis, blue) in untreated control mice. Data is log10-transformed; n=13 mice. k, Comparison of percent-positive of my-HSCs vs. bal-HSCs for CD47 (k, top) using two independent anti-CD47 clones (MIAP301, left; MIAP410, right), and for cKIT (k, bottom) using two independent anti-cKIT clones (ACK2, left; 2B8, right). Mouse ages were approximately: 4-6 months (a-i, k), or 3 -23 months (j). For a-k, bone-marrow was cKIT-enriched prior to FACS analysis. For j, total bone-marrow (non cKIT-enriched) was also examined. p-values and R values calculated with one-tailed Pearson correlation coefficient (i-j).

FIGS. 8A-8L. Identification of non-masking anti-CD150 antibodies. a, Schematic to identify and validate anti-CD150 antibodies that are not masked (e.g., not blocked) by anti-CD150 antibody clone 1 (TC15), used in panels b-d; bone-marrow cells were incubated with saturating concentrations (200 ug/mL) of unlabeled anti-CD150 antibody clone 1 (TC15) and then stained with PE anti-CD150 clones 2, 3, 4 (Q38, 9D1, mShad150). b-d, Saturating concentrations (200ug/mL) of unlabeled anti-CD150 antibody clone TC15 blocks staining with PE anti-CD150 clone 4 (mShad150) (d), but does not block staining of PE anti-CD150 clones 2, 3 (Q38, 9D1) (b-c). e, Schematic of experiment to determine if anti-CD150 clones 2, 3, 4 (Q38, 9D1, mShad150) identify the same population as anti-CD150 clone 1 (TC15); used in panels f-h; bone-marrow cells were incubated with PECy-7 anti-CD150 antibody clone 1 (TC15) and with PE anti-CD150 clones 2, 3, 4 (Q38, 9D1, mShad150). f-h, Co-staining with anti-CD150 clones 2, 3 (Q38, 9D1) identifies the same population as anti-CD150 antibody clone TC15 (f-g). Co-staining with anti-CD150 clone 4 (mShad150) and anti-CD150 antibody clone 1 (TC15) is mutually blocked (h). i, Schematic to identify anti-CD150 antibodies that are not blocked by anti-CD150 clone mShad150, used in panel j; bone-marrow cells are incubated with saturating concentrations (200 ug/mL) of unlabeled anti-CD150 antibody clone mShad150 and then stained with PE anti-CD150 clone 2 (Q38). j, Saturating concentrations (200 ug/mL) of unlabeled anti-CD150 antibody clone mShad150 does not block staining of PE anti-CD150 clone 2 (Q38) (j). k, Schematic of experiment to determine if co-staining with anti-CD150 clone 2 (Q38) identifies the same population as anti-CD150 antibody clone mShad150; used in panel I; bone-marrow cells are incubated with PECy-7 anti-CD150 antibody clone mShad150 and with PE anti-CD150 clone 2 (Q38). I, Co-staining with anti-CD150 clones 2 (Q38) identifies the same population as anti-CD150 antibody clone mShad150 (I). Mouse ages were approximately: 3 months (a-h) or 5-8 months (i-l). For (a-l), bone-marrow was cKIT-enriched prior to FACS analysis.

FIGS. 9A-9S. Antibody-mediated depletion of my-HSCs in vivo. a-b, Frequency as a percentage of live cells (a) or absolute number of cells (b) of my-HSCs and bal-HSCs after CD150 antibody conditioning (with anti-CD150, anti-CD150+anti-CD47, or anti-CD150+anti-CD47+anti-cKIT); n=3 mice. c-d, Frequency as a percentage of live cells (c) or absolute number of cells (d) of NEO1⁺HSCs and NEO1-HSCs after CD150 antibody conditioning (with anti-CD150, anti-CD150+anti-CD47, or anti-CD150+anti-CD47+anti-cKIT); n=3 mice. e, Total HSCs (e.g., my-HSCs+bal-HSCs) as a percentage of live cells, in mice receiving anti-CD47 alone; n=5 mice. f, Percentage of total HSCs that are my-HSCs in mice receiving anti-CD47 alone; n=5 mice. g, Total HSCs (e.g., my-HSCs +bal-HSCs) as a percentage of live cells, in mice receiving anti-CD47+anti-cKIT; n=4 mice. h, Percentage of total HSCs that are my-HSCs in mice receiving anti-CD47+anti-cKIT; n=4 mice. i, Percentage of total HSCs that are NEO1⁺ HSCs in mice receiving anti-CD150 (of IgG2a isotype)+anti-CD47+anti-cKIT, (protocol CD150^{v2}); n=4 mice. j-n, Frequency as a percentage of live cells for CLPs (j), IL7Ra⁺ cells (k), CMPs&GMPs (I), MkPs (m), and MEPs (n), after CD150, CD62p, or NEO1 antibody-conditioning protocols. Values are relative to the mean of control mice and log2-transformed; n=3 mice (NEO1^{v1}, NEO1^{v2}, CD150^{v1}); n=4 mice (CD62p, CD150^{v2}). o, Ratio of the frequency of live cells for Lymphoid to Myeloid Progenitors (CLP)/(CMP&GMP), after CD150, CD62p, or NEO1 antibody conditioning protocols. Values are relative to the mean of control mice and log2-transformed; n=3 mice (NEO1^{v1}, NEO1^{v2, CD}150^{v1); n=}4 mice (CD62p, CD150^{v2}). p, Frequency of my-HSCs, bal-HSCs, NEO1⁺HSCs, and NEO1-HSCs as a percentage of live cells after treatment with anti-CD150, anti-CD62p, or anti-NEO1 optimized conditioning protocols (e.g., +anti-CD47, +anti-cKIT); n=3 mice (anti-CD150^{v1}); n=4 mice (anti-CD62p); n=6 mice (anti-NEO1, combined 90 ug&200 ug protocols). Values are relative to the mean value for control mice not receiving antibody-conditioning and log2-transformed. For j-p, values for treated mice are in closed filled circles; values for control mice are in open unfilled circles. q, Correlation of the fraction of live cells for my-HSCs vs. NEO1⁺ HSCs (left), and bal-HSCs vs. NEO1-HSCs (right), of control mice and mice receiving anti-CD150, anti-CD62p, or anti-NEO1 optimized conditioning protocols, in cKIT-enriched bone-marrow; n=3 mice (anti-CD150^{v1}); n=4 mice (anti-CD62p); n=6 mice (anti-NEO1, combined 90 ug&200 ug protocols). r, Comparison of the absolute number of cells in total (non-cKIT-enriched) bone-marrow (y-axis) vs. frequency of cells as a percentage of live cells (in cKIT-enriched) bone-marrow (x-axis), for my-HSCs, bal-HSCs, CLP, CMP&GMP, and MkP in control mice and mice receiving anti-CD150, anti-CD150+anti-CD47, or anti-CD150+anti-CD47+anti-cKIT; n=3 mice per condition. s, Comparison of the frequency of cells as a fraction of total (non-cKIT-enriched) bone-marrow (x-axis) vs. the frequency of cells as a fraction of cKIT-enriched bone-marrow (y-axis), for numerous cell populations (KLS, total HSC, my-HSC, bal-HSC, NEO1⁺HSC, NEO1⁻HSC, OPP, CMP&GMP, MEP, CLP) in control mice and mice receiving anti-CD150, anti-CD150+anti-CD47, or anti-CD150+anti-CD47+anti-cKIT; n=3 mice per condition. Data for graphs in q-s are log 10-transformed. Mouse ages were approximately: 6-7 months (a-d, r-s), 7-9 months (g-i), or 5-9 months (j-q). For (a, c, g-s), bone-marrow was cKIT-enriched prior to FACS analysis. For (b, d, e-f, r-s), total bone-marrow (non cKIT-enriched) was examined. p-values were obtained by ordinary one-way ANOVA followed by one-tailed Dunnett's multiple comparisons test with non-treated as control (a-d), or by unpaired parametric one-tailed t-test (i-k, o-p), or by unpaired parametric two-tailed t-test (e-h, l-n). p-values and R values calculated with one-tailed Pearson correlation coefficient (q-s). CD150^{v1} is rat IgG2b anti-CD150 protocol; CD150^{v2} is rat IgG2a anti-CD150 protocol; NEO1^{v2} is protocol including mouse IgG2a secondary antibody; a, anti-; ns, not significant.

FIGS. 10A-10Q. Optimization of NEO1 depletion protocol in vitro and in vivo. a-f, Anti-NEO1 antibody saturation curve (a) determined from in vitro antibody concentration dilution series (b-f). g, Schematic of in vivo saturation experiments with anti-NEO1 antibody; used in panels h-k. h-i, Dose-dependent relationship between anti-NEO1 antibody dose (0 ug, 30 ug, 90 ug, 200 ug), when combined with anti-CD47 and anti-cKIT, on the relative depletion of NEO1⁺HSCs (h), and increase in CLPs (i). Optimal concentration highlighted in yellow; n=3 mice. j, The increase in NEO1⁻HSCs observed after anti-NEO1 dose escalation (0 ug, 30 ug, 90 ug, 200 ug) is correlated with the increase in CLPs. k, Impact on the ratio of Bal-HSCs/My-HSCs (black) and NEO1⁻HSCs/NEO1⁺HSCs (blue) as a percentage of live cells after anti-NEO1 antibody dose-escalation (0 ug, 30 ug, 90 ug, 200 ug), when combined with anti-CD47 and anti-cKIT; n=3 mice. Values are relative to the mean value of control (0 ug) mice and log2-transformed. I, Schematic illustrating paradigm for double-antibody strategy to target NEO1,whereby mouse monoclonal anti-goat IgG 2a or IgG 2b antibodies are administered 24 hours after goat anti-NEO1. m-n, Schematic (m) of experiment to demonstrate that saturating concentrations of mouse anti-goat IgG2a or IgG2b do not reduce ability of donkey anti-goat AF488 detect goat anti-NEO1 antibody (n). o-q, Schematic (o) of experiment demonstrating that mouse anti-goat IgG2a A555 (p) and IgG2b PE (q) antibodies identify the same population as donkey anti-goat AF488 by flow-cytometry. Mouse ages were approximately: 5-7 months (a-f, l-q) or 6-9 months (g-k). For (a-q), bone-marrow was cKIT-enriched prior to FACS analysis. For correlation p-values, one-tailed Pearson correlation coefficient (Rₚ), and one-tailed Spearman correlation coefficient (Rs) were calculated (j). p-values were obtained by ordinary one-way ANOVA followed by one-tailed Dunnett's multiple comparisons test with 0 g condition as control (k). MaG, mouse-anti goat; DaG, donkey anti-goat.

FIGS. 11A-11N. My-HSC depletion restores features of a youthful immune system. a, Ratio of the frequency of my-HSCs to bal-HSCs as a percentage of live cells in aged (A), or aged+conditioning mice (A+C) after approximately 8-weeks; n=4 mice (A), n=9 mice (A+C). b-d, Volcano plots of statistical significance (y-axis,-log10p) vs. fold-change (x-axis, log2) for Aged/Young (b), Aged/Aged+Conditioning (c), or Aged/(Young & Aged+Conditioning) (d), mice comparison. Gray bar set from y=1 to y=1.3; values above gray bar are p<0.05; n=6 mice (Young, Aged), n=5 mice (Aged+Conditioning). e, Overlap of top 17% of plasma proteins, ranked by statistical significance, increased in Aged/Young mice comparison and decreased in Aged+Conditioning/Aged mice comparison. f, Estimated plasma concentration of CXCL5 in Young (Y), Aged (A), or Aged+Conditioning mice (A+C); n=6 mice (Y, A), n=5 mice (A+C). g, Relative plasma abundance of a pre-defined set of inflammatory proteins in young (Y), aged (A), and aged+conditioning (A+C) mice; n=6 mice (Y, A), n=5 mice (A+C). Values are relative to the mean value for young mice (Y) and log2-transformed. h-i, Frequency of CD11b⁺Ly6G/C⁺ (h) and CD11b⁺SIRPα⁺ (i) mature myeloid cells in the blood of young-adult (Y), aged (A), or aged+conditioning mice (A+C) mice after approximately 1-week; n=3 mice (A), n=4 mice (A, A+C). j-k, Frequency of mature B cells (B220⁺CD19⁺CD43⁻CD93⁻IgM⁺IgD⁺) (j), and progenitor B cells (B220⁺CD19⁻ B cells) (k), in the bone-marrow of young-adult (Y), aged (A), or aged+conditioning mice (A+C) mice after approximately 1-week; n=5 mice (A), n=6 mice (A, A+C). Thymus weight (I) and frequency of thymic subsets 1-8 as defined by Akashi & Weissman, as a percentage of total CD45⁺ cells in the thymus (n), for young-adult (Y), aged (A), or aged+conditioning mice (A+C) mice after approximately 8-weeks, with representative flow-cytometry (m); n=3 mice (Y, A), n=9 mice (A+C). Populations enriched for transitional intermediate cells (areas 3&4) are highlighted in enclosed box (n). Mouse ages are at time of analysis: Y, young-adult (3-6 months) mice; A, aged (18-24 months) mice; A+C, aged (18-24 months) mice receiving antibody-conditioning. For (a), bone-marrow was cKIT-enriched prior to analysis. p-values were obtained by unpaired parametric one-tailed t-test (a), by ordinary one-way ANOVA followed by one-tailed Dunnett's multiple comparisons test using Aged as control (f), by one-way ANOVA followed by Holm multiple comparisons test (b-d, g), by ordinary one-way ANOVA followed by two-tailed Dunnett's multiple comparisons test using Aged as control (h-i, l, n), or by Brown-Forsythe and Welch ANOVA tests followed by Dunnett's T3 multiple comparisons test using Aged as control (j-k).

FIGS. 12A-12J. My-HSC depletion increases naïve T cells and B cells in aged mice. a-c, Absolute numbers of (a) naïve (CD44⁻CD62L⁺), (b) central memory (CD44⁺CD62L⁺), or (c) effector memory (CD44⁺CD62L⁻) T cells (combined CD4 & CD8), per mL of blood in aged (A) and aged mice receiving antibody-conditioning (A+C), approximately 8-weeks post-treatment. Values are log2-transformed; n=9 mice (A), n=14 mice (A+C), pooled from 2 independent experiments. d, Absolute numbers of mature B cells (IgM+IgD+) per mL of blood in aged (A), and aged mice receiving antibody-conditioning (A+C), approximately 8-weeks post-treatment. Values are log2-transformed; n=6 mice (A), n=5 mice (A+C). e, Absolute numbers of CD45+ cells per mL of blood in aged (A), and aged mice receiving antibody-conditioning (A+C), approximately 8-weeks post-treatment. Values are log2-transformed; n=3 mice (A), n=9 mice (A+C). f-g, Percentage of central memory (f, CM: CD44⁺CD62L⁺) and effector memory (g, EM: CD44⁺CD62L⁻) subsets per total T cells (combined CD4 & CD8) in aged mice receiving antibody-conditioning (A+C) and in young-adult mice (Y), relative to age-matched controls (A), 8-weeks post-treatment; n=6 mice (Y, A), n=5 mice (A+C). h-j, Frequency relative to aged mice of T cell (CD4 & CD8) subsets in Young (Y), Aged (A), and Aged+Conditioning (A+C) mice 8-weeks after antibody treatment (h). Naïve, central memory (CM), and effector memory (EM) subsets were defined by 12-marker cluster-based analysis (i-j); n=6 mice (Y, A), n=5 mice (A+C). Values are relative to the mean value for aged control mice and log2-transformed. Mouse ages are at time of antibody-conditioning: Y, young-adult (3-6 months) mice; A, aged (18-24 months) mice; A+C, aged (18-24 months) mice receiving antibody-conditioning. p-values were obtained by unpaired parametric one-tailed t-test (a, d), by unpaired parametric two-tailed t-test (b-c, e), or by ordinary one-way ANOVA followed by two-tailed (f-g) or one-tailed (h) Dunnett's multiple comparisons test using Aged as control.

FIGS. 13A-13L. Flow-cytometry gating strategy for T cells, B cells, and myeloid cells. a-c, Gating strategy to identify: (b) naïve (CD44⁻CD62L⁺), central memory (CD44⁺CD62L⁺), and effector memory (CD44⁺CD62L⁻) T cells (combined CD4 & CD8), or (c) CD4 T cells that are PD1⁺CD62L⁻ or PD1⁻CD62L⁺, in the blood. d-f, Gating strategy to identify: (e) mature B cells (CD19⁺B220⁺IgM⁺IgD⁺), or (f) Aged B Cells ABCs (CD19⁺IgM⁺CD93⁻CD43⁻CD21/CD35⁻CD23⁻), in the blood. g-i, Gating strategy to identify (h) progenitor B cells (B220⁺CD19⁻), or (i) mature B cells (B220⁺CD19⁺CD43⁻CD93⁻IgM⁺IgD⁺), in the bone-marrow. j-I, Gating strategy to identify (k) CD11b⁺Ly6G/C⁺ myeloid cells, or (l) CD11b⁺SIRPα⁺ myeloid cells, in the blood.

FIGS. 14A-14J. Antibody-conditioning enhances functional immunity to infection. a, Schematic of infectious disease model to determine the impact of antibody-conditioning on functional immunity of aged mice. Young-adult mice (Y) were compared to aged mice (A), with or without antibody conditioning with anti-NEO1 optimized protocol (A+C). Mice were vaccinated, or were not vaccinated, at Week-8 post-antibody conditioning, infected at Week-14, and analyzed at Week-16. b, Gating strategy to identify Ter119⁺ cells (Ter119⁺CD19⁻CD3⁻CD45^{+/lo}) and antigen-infected cells (Ag34⁺Ter119⁺) in mouse spleens. c-h, My-HSC and NEO1⁺HSC absolute numbers in total bone-marrow (c, e), or as percentage of total HSCs (d, f), with correlations (h), in young-adult (Y), aged (A), or aged+conditioning mice (A+C) 10-weeks after anti-NEO1^{v2} conditioning protocol. Representative flow cytometry of NEO1 staining on total HSCs (g). Aged and aged+conditioning mice received vaccination at Week 8; n=2 mice (Y), n=7 mice (A), n=10 mice (A+C). i, Total number of Ter119⁺ cells per mouse spleen was evaluated in young-adult (Y), aged (A), or aged+conditioning mice (A+C) that were Naïve, Infected, or Vaccinated & Infected with FV. Representative flow-cytometry histogram plots for Ter119 expression, gated on all single cells. Each row represents an independent mouse. n=9 mice (Y, naïve), n=13 mice (Y, FV infected), n=13 mice (Y, vaccinated & FV infected), n=6 mice (A, naïve), n=10 mice (A, FV infected), n=8 mice (A, vaccinated & FV infected), n=5 mice (A+C, naïve), n=9 mice (A+C, vaccinated & FV infected). Data is log10-transformed. Graph bars depict median. j, Total number of antigen-infected (Ag34⁺Ter119⁺) cells per spleen were measured in young-adult (Y), aged (A), or aged+conditioning mice (A+C) that were: Infected, or Vaccinated & Infected with FV. Representative flow-cytometry histogram plots for Ag34 expression, gated on Ter119+ cells. Each row represents an independent mouse; n=13 mice (Y, FV infected), n=13 mice (Y, vaccinated & FV infected), n=10 mice (A, FV infected), n=8 mice (A, vaccinated & FV infected), n=9 mice (A+C, vaccinated & FV infected). Data is log₁₀(x+1)-transformed. Graph bars depict median. For i-j, data from experiments using the anti-NEO1^{v1} conditioning protocol (open circles) or the anti-NEO1^{v2} conditioning protocol (closed circles) were combined. Mouse ages are at time of analysis: Y, young-adult (3-6 months) mice; A, aged (21-22 months) mice; A+C, aged (21-22 months) mice receiving antibody-conditioning. For (c-f), bone-marrow was cKIT-enriched prior to FACS analysis. For (c, e, h), total bone marrow (non-cKIT-enriched) was also analyzed to calculate total numbers of cells. p-values were obtained by ordinary one-way ANOVA followed by one-tailed Dunnett's multiple comparisons test using Aged as control (c-f), or by two-tailed Pearson correlation coefficient (h), or by ordinary one-way ANOVA followed by Tukey's multiple comparisons test (i-j). NEO1^{v2} protocol is NEO1^{v1} protocol (anti-NEO1+ anti-CD47+anti-cKIT)+mouse IgG2a secondary antibody. Y, young-adult mice; A, aged mice; A+C, aged mice receiving antibody-conditioning; Inf., FV infected without vaccination; Vacc. & Inf., FV infected with vaccination, Vacc. & Inf., FV infected with vaccination; LOD, limit of detection; n.s., not significant.

FIGS. 15A-15F. Mouse my-HSC markers are enriched in aged human HSCs. a, Relative mRNA expression of CD62p (Selp), CD41 (Itga2b), CD61 (Itgb3), CD150 (Slamf1), and NEO1 (Neo1) in human HSCs isolated from young (age 20-26) and old (age>70) donors. b, Relative mRNA expression of CD62p (Selp), CD41 (Itga2b), CD61 (Itgb3), and NEO1 (Neo1) in human HSCs isolated from young (age 18-30) or old (age 65-75) donors. c-e, Correlation of relative mRNA expression of CD62p (c), CD41 (d), and CD61 (e) in human HSCs as compared to donor age. For a-e, values are relative to mean of young samples. f, Heatmap depicting expression of candidate markers across independent datasets comparing human: HMGA2⁺ vs. HMGA2⁻CD34⁺ cells (e), MPN (f) or MDS (g) vs. normal HSCs, and Pre-AML vs. normal HSCs (h). p-values were obtained by unpaired parametric one-tailed t-test (a-b). For correlations, p-values and R values calculated with one-tailed Pearson correlation coefficient (c-e). MDS, myelodysplastic syndrome; MPN, myeloproliferative neoplasms.

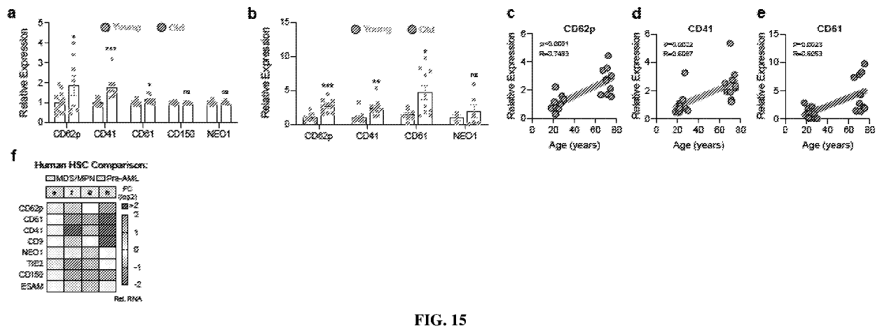

FIGS. 16A-16M. Mouse my-HSC antigens mark subsets of human HSCs. a, Representative flow-cytometry gating to identify human HSCs (Lin CD34⁺CD38⁻CD45RA⁻CD90⁺), MPPs (Lin⁻CD34⁺CD38⁻CD45RA⁻CD90⁻), LMPPs (Lin⁻CD34⁺CD38⁻CD45RA⁺), CMPs & MEPs (Lin⁻CD34⁺CD38⁺CD45RA⁻), and GMPs (Lin⁻CD34⁺CD38⁺CD45RA⁺), in normal human bone-marrow. Samples are post CD34⁺-enrichment. c-g, Flow-cytometry of human HSCs depicting fluorescence-minus-one (FMO) control (c), anti-CD62p clone AK4 (d), anti-CD62p clone Psel. KO2.3 (e), anti-CD62p clone AC1.2 (f), and anti-CD150 (g); representative for n=3 donors (d); n=2 donors (e-g). h, Illustration depicting human Hematopoietic Stem and Progenitor Cell (HSPC) Tree Analysis (h), with colors for each cell population corresponding to gating scheme in (a). i-l, Relative expression of CD62p (i), CD150 (j), ESAM (k), and CD166 (l), on human HSCs, MPPs, LMPPs, CMPs & MEPs, and GMPs. m, Percentage of positive HSCs and normalized MFI for each marker in HSCs and HSPCs for CD90, CD62p, TIE2, CD304, CD150, ESAM, CD166, CD105, CD47, and CD9. For i-m, Flow-cytometry median fluorescent intensity (MFI) values for each marker were obtained for each population, divided by the MFI for the FMO control, and then normalized from 0 -100 based on the lowest to highest expression. Red color scale corresponds to normalized MFI values. Blue, purple, maroon scale corresponds to bins for HSC positivity (20-50%, 51-70%, and 71-100).

### DETAILED DESCRIPTION OF THE EMBODIMENTS

It is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Hematopoietic stem cells (HSC). HSCs are functionally defined by their unique capacity to self-renew and to differentiate to produce all mature blood cell types. The term “HSC therefore refers to multipotent cells capable of differentiating into all the cell types of the hematopoietic system, including, but not limited to, granulocytes, monocytes, erythrocytes, megakaryocytes, lymphocytes, dendritic cells; and self-renewal activity, i.e. the ability to divide and generate at least one daughter cell with the identical (e.g., self-renewing) characteristics of the parent cell.

Human HSC are, for example, CD34⁺; CD90 (thy-1)⁺; CD59⁺; CD110 (c-mpl)⁺; c-kit (CD-117)⁺. A human HSC cell may be characterized or selected by the phenotype, for example, Lin⁻CD34⁺CD38⁻CD90⁺CD45RA⁻. Mouse HSC are, for example, CD90 (thy-1)^{lo}; Sca1⁺; c-kit (CD-117)⁺. A mouse HSC cell may be characterized or selected by the phenotype, for example, Lin⁻cKIT⁺Sca1⁺Flk2⁻CD34⁻CD150⁺. A “lin” or lineage panel may comprise one or more of the markers CD3, CD4, CD8, CD19, CD20, CD56, CD11b, CD14, and CD15.

In general, the process of development from pluripotent progenitors to mature cells with specific functions involves the progressive loss of developmental potential to other lineages. The earliest known lymphoid-restricted cell in adult mouse bone marrow is the common lymphocyte progenitor (CLP), and the earliest known myeloid-restricted cell is the common myeloid progenitor (CMP). A complete description of these cell subsets may be found in Akashi et al. (2000) Nature 404(6774):193, U.S. Pat. No. 7,300,760 (common myeloid progenitor); Kondo et al. (1997) Cell 91(5):661-7, U.S. Pat. No. 7,297,329 (common lymphoid progenitor); and is reviewed by Kondo et al. (2003) Annu Rev Immunol. 21:759-806, each of which is herein specifically incorporated by reference.

my-HSC and bal-HSC. A myeloid-biased HSC generates differentiated progeny with a greater proportion of myeloid progenitors, relative to a balanced HSC. my-HSCs can be defined by the ratio between lymphoid and myeloid cells in blood that are derived from the my-HSC. Balanced HSCs give rise to a blood population that is from about 10% to about 20% myeloid cells, with the remainder lymphocytes. The mean lymphold-to-myeloid cell ratio in the blood can be around 3.0±3.0. My-HSCs generate a mean lymphoid-to-myeloid cell ratio in the blood of less than about 3 but greater than 0. My-HSC generate myeloid and lymphoid progeny, but with an altered bias toward myeloid cells.

As shown herein, in addition to the functional distinctions, human my-HSC can be distinguished from bal-HSC by cell surface markers, including without limitation CD304, TIE2, ESAM, CD9, CD105, CD166, CD150 (Slamf1), CD61 (Itgb3), CD41 (Itga2b), CD62p, and NEO1, where these markers are expressed at higher levels on the my-HSC relative to the bal-HSC. The marker expression can be increased at least 1.5-fold, 2-fold, 3-fold, 5-fold, 10-fold or more on my-HSC relative to bal-HSC. In some embodiments my-HSC selective markers comprise one or more of CD150 (Slamf1), CD61 (Itgb3), CD41 (Itga2b), CD62p, and NEO1. In some embodiments my-selective markers are CD150, CD62p and NEO1.

Markers of mouse my-HSC include, for example, CD150, CD62p, NEO1, CD38, CD51 (Itgav), CD201 (Procr), CD202b (Tie2), ESAM (Esam), CD105 (Eng), and CD9.

Myeloid progenitor cells. Myeloid progenitor cells comprise one or more of: common myeloid progenitor cells (CMP); and the committed myeloid progenitors: erythroid/megakaryocytic progenitor (MEP), granulocyte/monocyte progenitors (GMP); and megakaryocyte progenitor (MKP).

Common Myeloid Progenitor (CMP) is hematopoietic progenitor subset that can give rise to all lineages of myeloerythroid cells, but lacks the potential to differentiate into lymphoid lineages. The CMP cells of both humans and mice stain negatively for the markers Thy-1 (CD90), IL-7Rα (CD127); and with a panel of lineage markers, which lineage markers may include CD2; CD3; CD4; CD7; CD8; CD10; CD11b; CD14; CD19; CD20; CD56; and glycophorin A (GPA) in humans and CD2; CD3; CD4; CD8; CD19; IgM; Ter110; Gr-1 in mice. The cells are CD34 positive, and CD38 positive. In humans, the CMP is also characterized as IL-3Rα^{lo} CD45RA⁻. In the mouse the CMP are Sca-1 negative, (Ly-6E and Ly-6A), c-kit^{hi}, and FcγR^{lo}.

Common lymphoid progenitors, CLP, express low levels of c-kit (CD117) on their cell surface. Antibodies that specifically bind c-kit in humans, mice, rats, etc. are known in the art. Alternatively, the c-kit ligand, steel factor (SIf) may be used to identify cells expressing c-kit. The CLP cells express high levels of the IL-7 receptor alpha chain (CDw127). Murine CLPs express low levels of Sca-1 (Ly-6E and Ly-6A, see van de Rijn (1989) Proc Natl Acad Sci 86:4634-4638). Human CLPs express low levels of CD 34. Human CLP cells are also characterized as CD38 positive and CD10 positive.

The CLP subset also has the phenotype of lacking expression of lineage specific markers, exemplified by B220, CD4, CD8, CD3, Gr-1 and Mac-1. The CLP cells are characterized as lacking expression of Thy-1, a marker that is characteristic of hematopoietic stem cells. The phenotype of the CLP may be further characterized as Mel-14, CD43^{lo}, HSA^{lo}, CD45⁺ and common cytokine receptor y chain positive.

Aged. As used herein, the term aged refers to the effects or the characteristics of increasing age, particularly with respect to the bias of hematopoietic stem cells towards cells of the myeloid lineage. The rate of aging is species specific, where a human may be aged at about 50 years; and a rodent at about 2 years. In general terms, a natural progressive decline in body systems starts in early adulthood, but it becomes most evident several decades later. One arbitrary way to define old age more precisely in humans is to say that it begins at conventional retirement age, around about 60, around about 65 years of age. Another definition sets parameters for aging coincident with the loss of reproductive ability, which is around about age 45, more usually around about 50 in humans, but may, however, vary with the individual. In addition to chronologic aging, individuals may suffer from a similar phenotype due to inflammation, genetic causes, and the like.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies. The term “entire” antibody is used to refer to an antibody comprising both variable regions and constant regions, i.e. an Fc region.

Selection of antibodies for stem cell depletion may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies. The cross-linking of Fc receptors signals the effector cells to kill the target cells by cytolysis or apoptosis. In one embodiment, the induction is achieved via antibody-dependent cellular cytotoxicity (ADCC).

“Affinity” refers to the strength of binding, increased binding affinity being correlated with a lower KD. In an embodiment, affinity is determined by surface plasmon resonance (SPR), e.g. as used by Biacore systems. The affinity of one molecule for another molecule is determined by measuring the binding kinetics of the interaction, e.g. at 25 °C

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Antibodies of interest may be tested for their ability to induce ADCC (antibody-dependent cellular cytotoxicity). Antibody-associated ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.). Preferably, the antibodies of the present invention induce at least 10%, 20%, 30%, 40%, 50%, 60%, or 80% cytotoxicity of the target cells.

In some embodiments, the antibody is conjugated to an effector moiety. The effector moiety can be any number of molecules, including labeling moieties such as radioactive labels or fluorescent labels, or can be a cytotoxic moiety. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, saporin, auristatin-E and the like. Cytotoxic agents also include radiochemicals made by conjugating radioisotopes to antibodies. Targeting the cytotoxic moiety to transmembrane proteins serves to increase the local concentration of the cytotoxic moiety in the targeted area.

Agents of interest that bind to CD150 include antibodies specific for human CD150. Such antibodies are known in the art and are commercially available, for example and without limitation SLAMF1/SLAM/CD150 Antibody LS-C204428; A12 monoclonal antibody; SLAM.4; and Clone REA151.

Agents of interest that bind to CD62p (P-selectin) include antibodies specific for human CD62p. Such antibodies are known in the art and commercially available, for example and without limitation inclacumab; Crizanlizumab; HuEP5C7; Clone REA389; clone AK-6; clone Psel. KO.2.12; etc.

Agents of interest that bind to NEO1 include antibodies specific for human NEO1. Such antibodies are known in the art and commercially available, for example and without limitation Neogenin Antibody (RM0124-3G55), Neogenin Antibody (221519), Neogenin Antibody (AF1079), etc.

Agents of interest that specifically bind to CD117 include antibodies that specifically bind to human CD117, and c-kit ligand. CD117 is a receptor tyrosine kinase type III, which binds to stem cell factor (a substance that causes certain types of cells to grow), also known as “steel factor” or “c-kit ligand”. When this receptor binds to stem cell factor (SCF) it forms a dimer that activates its intrinsic tyrosine kinase activity, that in turn phosphorylates and activates signal transduction molecules that propagate the signal in the cell. See, for example, the human refseq entries Genbank NM_000222; NP_000213. CD117 is an important cell surface marker used to identify certain types of hematopoietic (blood) progenitors in the bone marrow. Hematopoietic stem cells (HSC), multipotent progenitors (MPP), and common myeloid progenitors (CMP) express high levels of CD117. A number of antibodies that specifically bind human CD117 are known in the art and commercially available, including without limitation SR1, 2B8, ACK2, YB5-B8, 57A5, 104D2, etc. Of interest is the humanized form of SR1, AMG 191, described in U.S. Pat. Nos. 8,436,150, and 7,915,391 which is an aglycosylated IgG1 humanized antibody.

As used herein, the term “anti-CD47 agent” or “agent that interferes with the binding between CD47 and SIRPα” refers to any agent that reduces the binding of CD47 (e.g., on a target cell) to SIRPα (e.g., on a phagocytic cell). Non-limiting examples of suitable anti-CD47 reagents include high affinity SIRPα polypeptides, anti-SIRPα antibodies, and anti-CD47 antibodies or antibody fragments. In some embodiments, a suitable anti-CD47 agent (e.g. an anti-CD47 antibody, a SIRPα reagent, etc.) specifically binds CD47 to reduce the binding of CD47 to SIRPα.

Anti-human CD47 antibodies suitable for clinical use include, without limitation, magrolimab (hu5F9-G4, see U.S. Pat. No. 9,017,675), AK117; AO-176; CC-90002; IBI188; IMC-002; SHR-1603; SRF231; STI-6643; TJ011133; and ZL-1201. Soluble SIRPα agents include, for example, Evorpacept (ALX148), and CV1-Fc (see, for example, Weiskopf et al. (2013) Science 341 (6141): 88-91). Such antibodies may comprise an Fc region sequence.

In some embodiments, an anti-CD47 agent is a “high affinity SIRPα reagent”, which includes SIRPα-derived polypeptides and analogs thereof (e.g., CV1-hIgG4, and CV1 monomer, ALX148). High affinity SIRPα reagents are described in international application PCT/US13/21937, which is hereby specifically incorporated by reference. High affinity SIRPα reagents are variants of the native SIRPα protein. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, etc. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

Optionally a SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

In some embodiments, a subject anti-CD47 agent is an antibody that specifically binds SIRPα (i.e., an anti-SIRPα antibody) and reduces the interaction between CD47 on one cell (e.g., an infected cell) and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Anti-SIRPα antibodies in clinical and preclinical trials for human use include, for example, CC-95251; BYON4228; SIRPα-targeting antibody BR105; BI 770371 and BI-765063/OSE172 (Boehringer Ingelheim); and GS-189 (FSI-189) (Gilead Sciences).

A “patient” for the purposes of the present invention includes both humans and other animals, particularly mammals, including pet and laboratory animals, e.g. mice, rats, rabbits, etc. Thus, the methods are applicable to both human therapy and veterinary applications. In one embodiment the patient is a mammal, preferably a primate. In other embodiments the patient is human.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already affected (e.g., those with cancer, those with an infection, etc.) as well as those in which prevention is desired (e.g., those with increased susceptibility to cancer, those with an increased likelihood of infection, those suspected of having cancer, those suspected of harboring an infection, etc.).

### Selective Depletion

Methods of selective depletion of my-HSC provide for an improved balance in the levels of myeloid versus lymphoid cells in a subject after depletion. The recipient is conditioned with the administration of an effective dose of conditioning agents, e.g. an antibody, specific for a my-HSC selective marker, or a combination of my-HSC selective agents. The my-HSC selective agent(s) may be combined with one or both of an agent that blocks CD47 interaction with SIRPα, and an agent that specifically binds to CD117.

The dose of agents administered to a subject is effective to provide for selective depletion, which enriches the population of bal-HSC to my-HSC in the subject by at least 1.5-fold, at least 2-fold, at least 3-fold, at least 5-fold, at least 7.5-fold, at least 10-fold, at least 15-fold, or more. After a period of time sufficient for rebalancing, e.g. after about 1 week, after about 2 weeks, after about 3 weeks, the ratio of the number of lymphoid progenitors in bone marrow, e.g. common lymphoid progenitors, to the number of myeloid progenitors, e.g. common myeloid progenitors, may be increased at least 1.5-fold, at least 2-fold, at least 3-fold, at least 5-fold, at least 7.5-fold, at least 10-fold, at least 15-fold, or more. The number of circulating naïve T cells relative to the total circulating lymphocyte population may be increased at least 1.5-fold, at least 2-fold, at least 3-fold, at least 5-fold, at least 7.5-fold, at least 10-fold, at least 15-fold, or more. The level of circulating “age-associated B cells” (ABC), and/or exhausted T cells relative to the total circulating lymphocyte population may be decreased at least 1.5-fold, at least 2-fold, at least 3-fold, at least 5-fold, at least 7.5-fold, at least 10-fold, at least 15-fold, or more.

The effective dose of a my-HSC selective agent, e.g. antibody, will depend on the individual and the specific antibody, but will generally be at least about 50 μg/kg body weight, at least about 250 μg/kg, at least about 500 μg/kg, at least about 750 μg/kg, at least about 1 mg/kg, and up to about 2.5 mg/kg, up to about 5 mg/kg, up to about 7.5 mg/kg, up to about 10 mg/kg, up to about 15 mg/kg, up to about 25 mg/kg, up to about 50 mg/kg, up to about 100 mg/kg. In some embodiments an antibody is specific for human CD150. In some embodiments an antibody is specific for human CD62p. In some embodiments an antibody is specific for human NEO1.

The effective dose of an agent that specifically binds to CD117, e.g. an anti-CD117 antibody or stem cell factor, will depend on the individual and the specific agent. In some embodiments the agent is an antibody, and will generally be administered at a dose at least about 50 μg/kg body weight, at least about 250 μg/kg, at least about 500 μg/kg, at least about 750 μg/kg, at least about 1 mg/kg, and up to about 2.5 mg/kg, up to about 5 mg/kg, up to about 7.5 mg/kg, up to about 10 mg/kg, up to about 15 mg/kg, up to about 25 mg/kg, up to about 50 mg/kg, up to about 100 mg/kg.

The effective dose of an agent that blocks CD47 interaction with SIRPα, for example an anti-CD47 antibody, anti-SIRPα antibody, or soluble SIRPα polypeptide, will depend on the individual and the specific agent, but will generally be at least about 50 μg/kg body weight, at least about 250 μg/kg, at least about 500 μg/kg, at least about 750 μg/kg, at least about 1 mg/kg, and up to about 2.5 mg/kg, up to about 5 mg/kg, up to about 7.5 mg/kg, up to about 10 mg/kg, up to about 15 mg/kg, up to about 25 mg/kg, up to about 50 mg/kg, up to about 100 mg/kg. In some embodiments the agent is a CV1 (high affinity SIRPα) monomer or CV1 microbody dimer. In other embodiments the agent is an anti-CD47 antibody. In other embodiments the agent is an anti-SIRPα antibody.

The depleting agents can be administered daily, twice daily, every other day, every third day, etc. for a period of time sufficient to affect the desired selective depletion, which may be at least about 1 day, up to about 2 days, up to about 3, 4, 5, 6, 7, 8 or more days. In some embodiments from 4-7 days is sufficient. In some embodiments a single dose is administered. In other embodiments a plurality of doses is administered, e.g. 2, 3, 4, 5 or more. The agents may be formulated together or separately, but are administered concomitantly. “Concomitant” and “concomitantly” as used herein refer to the administration of at least two agents, or at least three agents, or more to a patient either simultaneously or within a time period during which the effects of the first administered agent are still operative in the patient. Thus, if the first drug is, e.g., anti-CD117 antibody and the second drug is a soluble SIRPα, the concomitant administration of the second agent can occur one to two days after the first, preferably within one to seven days, after the administration of the first agent.

The administration of the agents can be done in a variety of ways as discussed above, including, but not limited to, orally, subcutaneously, intravenously, intranasally, transdermally, intraperitoneally, intramuscularly, intrapulmonary, vaginally, rectally, or intraocularly. Administration is preferably parenteral, e.g. intravenous.

The compositions containing depleting agents, e.g. antibodies, soluble SIRPα, etc. can be administered for therapeutic treatment. Compositions are administered to a patient in an amount sufficient to selectively deplete my-HSC, as described above. An amount adequate to accomplish this is defined as a “therapeutically effective dose.” Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### Formulations

For depletion, each of the agents is formulated in a pharmaceutical composition. The agents can be formulated separately or together, usually separately. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for patient condition, systemic versus localized delivery, as well as the age, body weight, general health, sex, diet, time of administration, drug interaction and the severity of the condition may be necessary, and will be ascertainable with routine experimentation by those skilled in the art.

In one embodiment, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly useful are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Compositions are administered to a patient in an amount sufficient to substantially deplete targeted myHSC, as described above. An amount adequate to accomplish this is defined as a “therapeutically effective dose.” Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### USES

The methods disclosed herein provide for a rebalancing of immune systems, generally to increase the production of lymphoid cells relative to myeloid cells. An imbalance is associated with aging and the elderly. By rebalancing, the ability of the individual to respond to novel pathogens is improved, for example to SARS-COV2, monkeypox, etc., and other pathogens not previously encountered by the subject. The ability of an individual to respond to vaccination is also improved.

In addition to improving the individual's adaptive immune responses, the expansion of myeloid-biased HSCs with age can contribute to aberrant immune inflammatory responses. Myeloid-biased HSCs are pro-inflammatory, producing or eliciting inflammatory cytokines (TNF-α, IL-1, IL-6, etc.), in response to microbes or endogenous antigens. The morbidity and mortality of elderly patients infected with new respiratory pathogens such as new strains of influenza and SARS-COV-2 is not only because of poor and delayed adaptive immune response, but also the inflammatory consequences. These heightened inflammatory responses driven by expansion of my-HSCs in the elderly also contribute to chronic inflammatory disease, which can occur in the absence of a pathogenic source. Rebalancing of the immune system through depletion of myeloid-biased HSCs allows for a more functional immune response, by increasing the generation of new T and B cells and reducing the production of inflammatory myeloid cells.

Selective depletion of myeloid-biased hematopoietic stem cells has relevance to blood and solid cancers. The diminution of adaptive immunity and the confounding presence of a more inflammatory milieu in the aged contributes to an inability to recognize and eliminate newly arising cancers. Rebalancing restores surveillance systems required for transformed and partially transformed cells that drive cancer, and reduces the generation of myeloid cells that suppress tumor immunity.

In some embodiments, the disclosure provides compositions and methods for use in a therapeutic method of rebalancing the immune system in a human subject in need thereof. These methods bring the body from a pathological state back into its normal, healthy state, or prevent a pathological state. In some embodiments, the disclosure provides compositions and methods for use in a therapeutic method of improved response to infection and/or vaccination. In some embodiments, the disclosure provides compositions and methods for use in a therapeutic method in reducing inflammation, e.g. inflammation associate with infection. In some embodiments, the disclosure provides compositions and methods for use in in a therapeutic method improving surveillance of cancer cells. In some embodiments, the disclosure provides compositions and methods for use in in a therapeutic method that reduces the population of myeloid cells that suppress tumor immunity.

In some embodiments, the disclosure provides compositions and methods for rebalancing the immune system of individuals suffering or at risk of a hematologic malignancy. Examples of hematologic malignancies and pre-malignancies that may be treated using the subject methods include leukemias, lymphomas, and myelomas, including but not limited to acute biphenotypic leukemia, acute myelogenous leukemia (AML), acute lymphoblastic leukemia (ALL), acute promyelocytic leukemia (APL), biphenotypic acute leukemia (BAL) blastic plasmacytoid dendritic cell neoplasm, chronic myelogenous leukemia (CML), chronic myelomonocytic leukemia (CMML), chronic lymphocytic leukemia (CLL) (called small lymphocytic lymphoma (SLL) when leukemic cells are absent), acute monocytic leukemia (AMOL), Hodgkin's lymphomas, Non-Hodgkin's lymphomas (e.g. chronic lymphocytic leukemia (CLL), diffuse large B-cell lymphoma (DLBCL), Follicular lymphoma (FL), Mantle cell lymphoma (MCL), Marginal zone lymphoma (MZL), Burkitt's lymphoma (BL), Hairy cell leukemia, Post-transplant lymphoproliferative disorder (PTLD), Waldenstrom's macroglobulinemia/lymphoplasmacytic lymphoma, hepatosplenic-T cell lymphoma, and cutaneous T cell lymphoma (including Sezary's syndrome)), multiple myeloma, myelodysplastic syndrome, and myeloproliferative neoplasms. In particular embodiments, the subject methods find utility in treatment of leukemias, e.g. acute biphenotypic leukemia, acute myelogenous leukemia (AML), acute lymphoblastic leukemia (ALL), acute promyelocytic leukemia, chronic myelogenous leukemia (CML), chronic myelomonocytic leukemia, chronic lymphocytic leukemia (CLL), acute monocytic leukemia (AMOL).

Individuals selected for treatment may include, for example, individuals diagnosed with CHIP, pre-malignant AML patients or MDS patients, naïve AML patients who are ineligible for standard induction chemotherapy or allogeneic hematopoietic cell transplant due to age and/or co-morbidities; previously untreated intermediate and high risk myelodysplastic syndrome (MDS) patients; and MDS patients who are relapsed and/or refractory to frontline hypomethylating agents.

A pre-malignancy or pre-leukemia condition of interest includes myelodysplastic syndrome (MDS), which is group of clonal hematopoietic stem cell disorders typified by peripheral cytopenia, dysplastic hematopoietic progenitors, a hypercellular or hypocellular bone marrow, and a high risk of conversion to acute myeloid leukemia. Symptoms are referable to the specific cell line most affected and may include fatigue, weakness, pallor (secondary to anemia), increased infections and fever (secondary to neutropenia), and increased bleeding and bruising (secondary to thrombocytopenia). Diagnosis is by blood count, peripheral smear, and bone marrow aspiration and biopsy. Treatment with venetoclax, azacitidine or decitabine may help; if acute myeloid leukemia supervenes, it is treated per the usual protocols.

The syndrome is unified by the presence of distinct mutations of hematopoietic stem cells, most frequently in genes involved in RNA splicing. Myelodysplastic syndromes are characterized by ineffective and dysplastic hematopoiesis and include the following: Refractory anemia: Anemia with reticulocytopenia; normal or hypercellular marrow with erythroid hyperplasia, and dyserythropoiesis; blasts ≤5% of nucleated marrow cells; Refractory anemia with ringed sideroblasts: Same as refractory anemia with reticulocytopenia, except that ringed sideroblasts are >15% of nucleated marrow cells; Refractory cytopenia with multilineage dysplasia: Cytopenia not restricted to red cells; prominent dysplasia of white cell precursors and megakaryocytes; Refractory cytopenia with multilineage dysplasia and ringed sideroblasts: With ringed sideroblasts that are >15% of nucleated marrow cells; Refractory anemia with excess blasts (RAEB): Cytopenia of ≥2 cell lines with morphologic abnormalities of hematopoietic cells; hypercellular marrow with dyserythropoiesis and dysgranulopoiesis; blasts 5 to 9% (RAEB-I) or 10 to 19% (RAEB-II) of nucleated marrow cells; Myelodysplastic syndrome, unclassified: MDS that does not fall into any defined category; MDS with isolated del(5q): Typically severe anemia and thrombocytosis, with deletion of the long arm of chromosome 5; Chronic myelomonocytic leukemia (CMML) and juvenile myelomonocytic leukemia (JMML): Mixed myelodysplastic/myeloproliferative neoplasms; absolute monocytosis (>1000/mcL [>1/L]) in blood; significant increase in marrow monocyte precursors; Chronic neutrophilic leukemia: Characterized by neutrophilia and absence of the Philadelphia chromosome and the BCR-ABL1 fusion gene.

Risk of MDS increases with age due to the acquisition of somatic mutations that can promote clonal expansion and dominance of a particular hematopoietic stem cell, and possibly due to exposure to environmental toxins such as benzene, radiation, and chemotherapeutic agents (particularly long or intense regimens and those involving alkylating agents, hydroxyurea, and/or topoisomerase inhibitors). Chromosomal abnormalities (eg, deletions, duplications, structural abnormalities) are often present.

A condition of particular interest for treatment is clonal hematopoiesis of indeterminate potential (CHIP), which is a pre-malignant expansion of mutated blood stem cells. Hematopoietic stem cells (HSCs) randomly accumulate somatic mutations during aging. While most of these mutations have no consequence, rare fitness-increasing mutations may allow an HSC to clonally expand. This age-associated expansion is termed clonal hematopoiesis of indeterminate potential (CHIP). It is found in 10-30% of those older than 70, most commonly occurs due to mutations in transcriptional regulators such as DNMT3A, TET2, and ASXL1, and can be detected by sequencing of peripheral blood or bone marrow cells. These mutations are thought to provide a selective advantage to the hematopoietic stem cells in which they occur, and are detectable as clones in peripheral blood samples because the mutated stem cells maintain the ability to differentiate into circulating granulocytes, monocytes, and lymphocytes.

While CHIP is a pre-malignant expansion of mutated blood stem cells that also associates with non-hematological disorders, these mutations are also founding mutations for hematological neoplasms such as acute myeloid leukemia, it is unsurprising that CHIP associates with increased risk of developing these cancers. CHIP also associates with increased risk of atherosclerotic cardiovascular disease and death due to non-malignant causes.

The presence of CHIP can be determined by methods known in the art, for example by analyzing a patient sample(s) comprising hematopoietic cells. The cells can be isolated from a bone marrow, blood or blood-derived sample. A plurality of cells in the sample(s) are analyzed for the presence of clonality, usually by high throughput sequencing of polynucleotides isolated from the cell, for example whole exome sequencing, targeted sequencing of frequently mutated genes, etc. The number of cells analyzed may be at least 10², at least 10³, at least 10⁴, at least 10⁵ or more. The sequencing can be performed on bulk blood cells, e.g. PBLs, or on selected cell populations, e.g. myeloid cells, stem and progenitor cells, etc. The presence of CHIP can be defined by the presence of somatic mutations, where the most frequently mutated genes include, for example, DNMT3A, TET2, ASXL1, SF3B1, and GNB1. The variant allele fraction (VAF) can be determined, i.e. as the fraction of alleles present in the plurality of cells that comprise a specific somatic mutation. An individual is determined to be a CHIP carrier if the VAF is ≥0.08, >0.09, >0.1, >0.125, >0.15, >0.175, >0.2 or more. In some embodiments a cut-off of a VAF≥0.2 is used to define an individual as having CHIP. The data can be compared to measurements from a control normal cell population. The data can be normalized for comparison.

In some embodiments a kit is provided, comprising an effective dose of the one or more agents disclosed herein for selective depletion of my-HSC.

### EXPERIMENTAL

### Example 1

### Rejuvenating the Immune System by Depleting Myeloid-Biased Hematopoietic Stem Cells

Aging of the hematopoietic system is characterized by decreased lymphopoiesis and adaptive immunity, and increased inflammation and myeloid pathologies. Age-related changes in the function of hematopoietic stem cells (HSCs), which generate all blood cells throughout life, are thought to underlie these phenomena. During early life, HSCs with balanced output of lymphoid and myeloid cells predominate over HSCs with myeloid-biased output, thereby promoting the lymphopoiesis required for adaptive immune responses, while limiting the production of pro-inflammatory myeloid cells. In contrast, aging is associated with increased proportions of myeloid-biased HSCs resulting in decreased lymphopoiesis and increased myeloid cell-driven inflammation. Whether these age-related changes to HSCs can be reversed to restore youthful immune function is unclear. Here we demonstrate that antibody-mediated depletion of myeloid-biased HSCs (my-HSCs) in aged mice restores characteristic features of a youthful immune system, including increasing common lymphocyte progenitors (CLPs), naïve T cells, and B cells, while decreasing age-related markers of inflammation and immune decline. The targeted my-HSC antigens are also enriched in aged human HSCs, nominating them as therapeutic targets to rejuvenate the immune system in humans.

Importantly, we also demonstrate that antibody-mediated depletion of my-HSCs in aged mice improves vaccination responses and enhances vaccine-induced protection from viral infection. The targeted my-HSC antigens identified in this study are also enriched in aged human HSCs, where they are therapeutic targets to rejuvenate the immune system in humans.

A single hematopoietic stem cell (HSC) can generate all blood cells and self-renew to maintain the stem cell pool throughout life. HSCs demonstrate functional heterogeneity and can differ in their contribution to the lymphoid and myeloid cell lineages. At least two HSC subsets exist: (i) balanced HSCs (bal-HSC) that provide balanced production of lymphoid and myeloid cells, and (ii) myeloid-biased HSCs (my-HSC) that are biased towards predominant production of myeloid cells. The frequency of my-HSCs relative to bal-HSCs increases with age. This age-related shift from bal-HSCs to my-HSCs decreases lymphopoiesis and increases myelopoiesis, thereby contributing to numerous pathologies of the elderly, including reduced adaptive immunity, ‘inflammaging’, and several myeloid-related diseases. To address these and other age-related pathologies, we sought to develop a therapy to return the immune system to a more youthful state characterized by less myeloid-biased HSCs and more HSCs with balanced production of lymphoid and myeloid lineage cells.

We hypothesized therapeutic depletion of my-HSCs, the reduction of my-HSCs would enable bal-HSCs to reverse age-related immune decline by restoring lymphopoiesis and limiting myeloid cell-driven inflammation. My-HSCs have been demonstrated to express distinct cell-surface markers compared to bal-HSCs. Thus, we speculated that my-HSCs could be specifically targeted for depletion with antibodies to these, or to other, my-HSC specific markers. To this end, we identified my-HSC-specific antigens, depleted my-HSC in vivo, and characterized the impact of depletion on the hematopoietic system and immune phenotypes, including functional immunity to new infections.

### Results

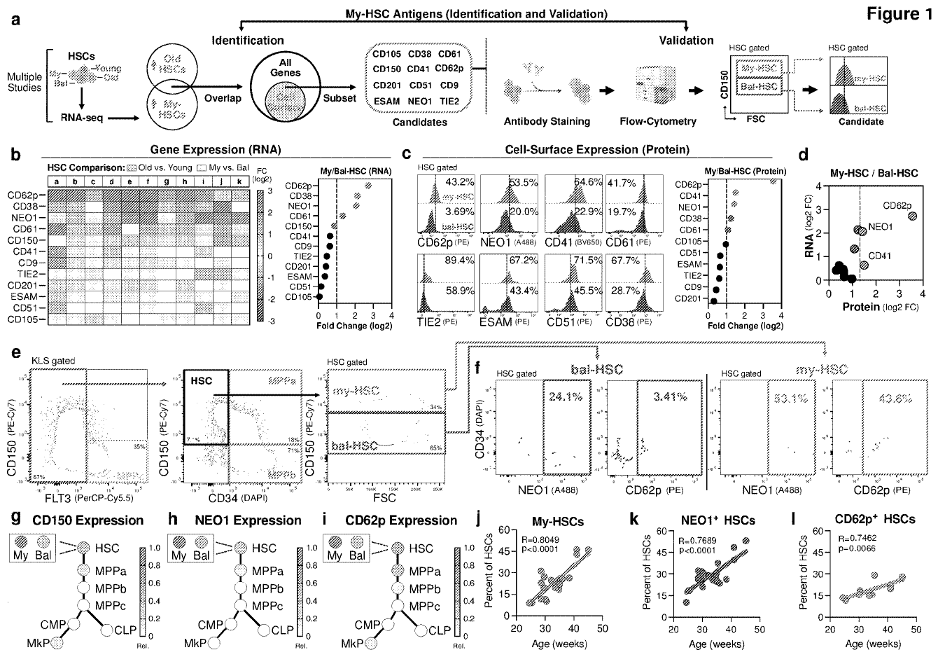

Identification of cell-surface antigens to deplete myeloid-biased hematopoietic stem cells. To identify candidate targets for therapeutic depletion of my-HSCs, we first established and validated a set of cell-surface antigens on my-HSCs (FIG. 1a). Mouse HSCs (Lin⁻cKIT⁺Sca1⁺FLT3⁻CD34⁻-CD150⁺) can be separated into my-HSCs or bal-HSCs based on their expression levels of CD150 (Slamf1); my-HSCs are CD150^{High}, while bal-HSCs are CD150^{Low}. In addition to CD150, several markers of HSCs with myeloid bias have been described. To identify the best target to deplete my-HSCs, we conducted a systematic search of all potential antigens increased relatively or absolutely on my-HSCs. My-HSCs are more abundant in aged animals and in subpopulations of HSCs defined by a combination of markers and/or genetic reporters. Thus, we reasoned that my-HSC specific genes would be enriched in transcriptional datasets of (i) HSCs from aged animals, and (ii) HSCs with functional myeloid-bias (FIG. 1b). Examination of these datasets yielded 12 candidate genes encoding cell-surface proteins that were highly enriched in aged and/or myeloid-biased HSCs (FIG. 1a-b). As expected, CD150 emerged from this analysis, along with several markers associated with myeloid-biased HSCs: CD41, CD61, CD62p, and NEO1. To validate these candidates and identify the best target on my-HSCs, we evaluated their cell-surface protein levels on my-HSCs and bal-HSCs with antibodies and flow-cytometry.

The ideal target antigen to deplete my-HSCs would be highly expressed on the cell-surface of my-HSCs relative to bal-HSCs. We compared the cell-surface expression of each candidate antigen on my-HSCs and bal-HSCs using marker-specific antibodies and flow-cytometry (FIG. 1c). For each marker, the fold-enrichment was calculated for my-HSCs based on the proportion of my-HSCs (CD150^{High} HSCs) relative to bal-HSCs (CD150^{Low} HSCs) that were marker-positive. Antibodies to NEO1 and CD41 resulted in a significantly increased frequency of staining of my-HSCs (FIG. 1c, 1f), consistent with NEO1 and CD41 marking HSCs with myeloid bias. Among the remaining candidates, CD62p led to the greatest enrichment for my-HSCs (FIG. 1c-f). Overall, the most highly enriched cell-surface proteins on my-HSCs relative to bal-HSCs were CD41, CD62p, and NEO1 (FIG. 1c-d). Together with CD150, we focused on these cell-surface proteins as candidate target antigens for antibody-mediated depletion of my-HSCs.

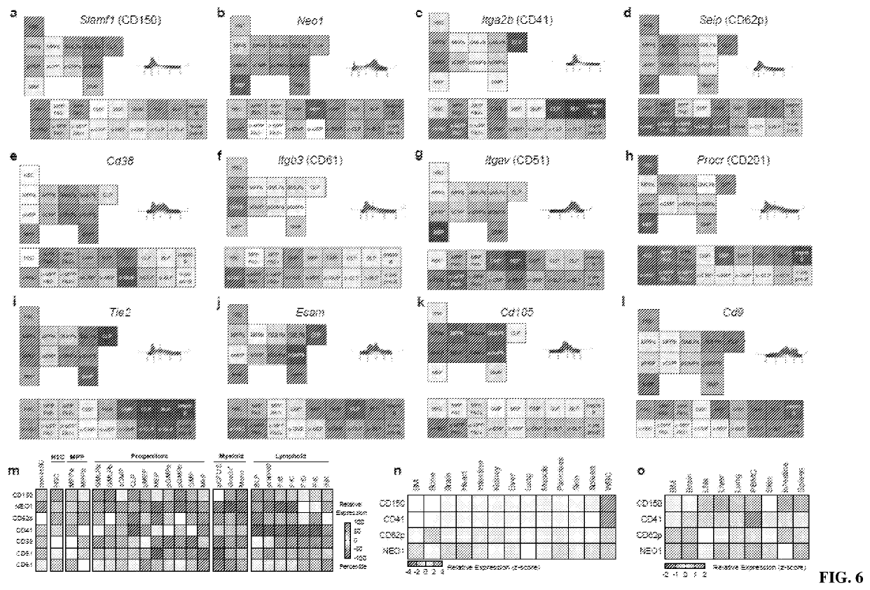

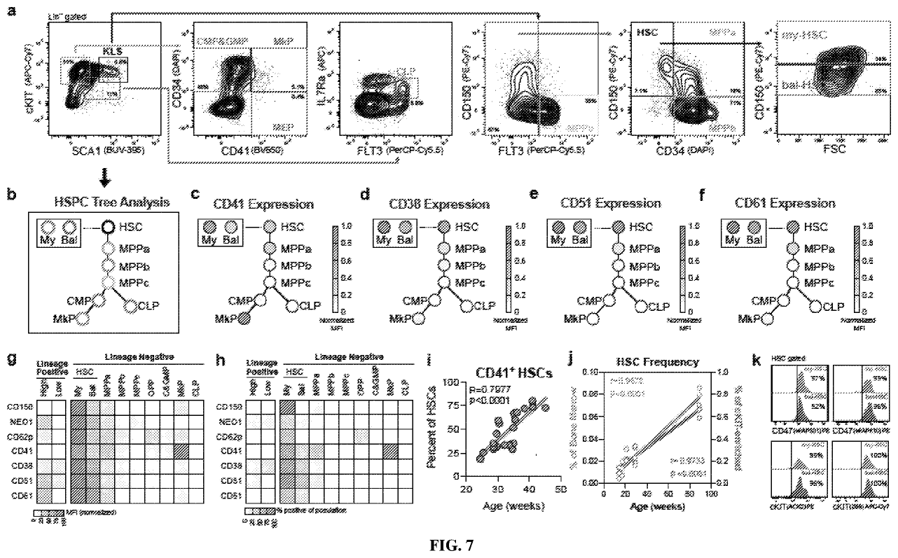

To provide insight into potential off-target effects from antibody treatment, we determined the expression of each candidate on hematopoietic progenitor cells (HPCs) and mature differentiated cells, as well as non-hematopoietic tissues. HSCs generate multi-potent progenitors (MPPa, MPPb, MPPc), which generate lineage-restricted common myeloid progenitors (CMPs) and common lymphoid progenitors (CLPs). Flow-cytometry revealed that none of the candidate surface proteins were highly expressed by these subsets, other than CD41, which was abundantly expressed in megakaryocyte progenitors (MkPs) (FIG. 1g-i and FIG. 7a-h). These results were largely concordant with transcriptomic profiling of these same sorted populations from independent datasets (FIG. 6a-l). The most promising candidates—CD150, CD41, CD62p, NEO1—were not highly expressed in mature hematopoietic cells by RNA (FIG. 6m) or by flow-cytometry (FIG. 7g-h) and were relatively specific to the hematopoietic system compared to other tissues (FIG. 6n-o). These results demonstrated the relative specificity of these markers to my-HSCs compared to bal-HSCs, hematopoietic progenitors and mature cells, and non-hematopoietic tissues.

Given the increased abundance of my-HSCs with age, we evaluated if HSCs isolated from aged mice demonstrated increased expression of the candidate markers we identified. We evaluated HSCs in a cohort of mice spanning approximately six months to one year of age, focusing on CD41, CD62p, and NEO1, which were the most highly enriched markers on my-HSCs. We observed a significant positive correlation with the proportion of my-HSCs and mouse age, consistent with the expansion of my-HSCs during aging (FIG. 1j and FIG. 7j). Similarly, we also observed a strong positive correlation with age and the percentage of total HSCs that were NEO1⁺, CD41⁺, or CD62p⁺ (FIG. 1k-l and FIG. 7i), consistent with NEO1, CD41, and CD62p marking my-HSCs that increase with age. We selected these cell-surface antigens, along with CD150, as candidate targets to deplete my-HSCs in vivo.

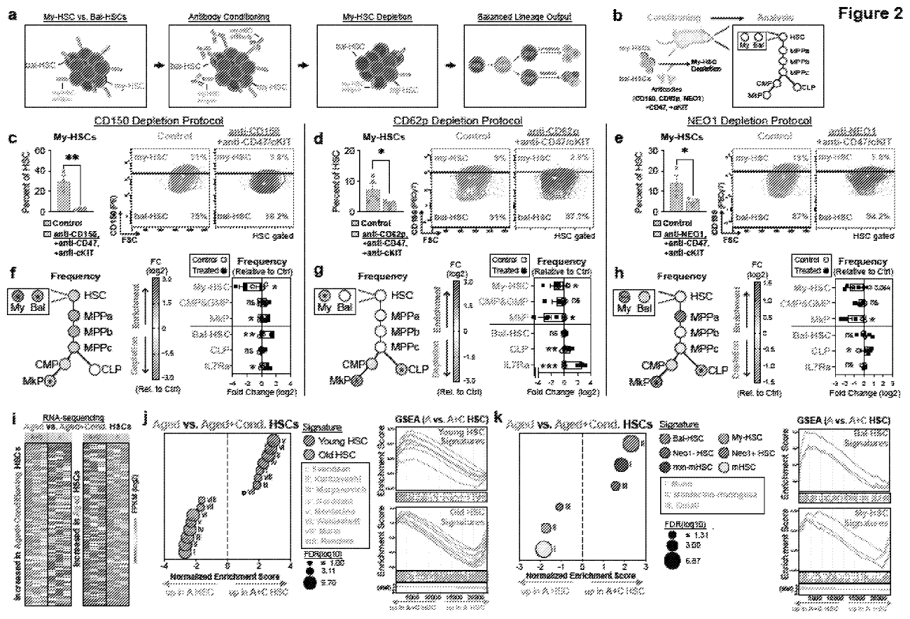

Antibody-mediated depletion of myeloid-biased hematopoietic stem cells in vivo. We next determined if we could deplete my-HSCs in vivo by targeting these my-HSC antigens (FIG. 2a). We focused on CD150, CD62p, and NEO1, which demonstrated the greatest enrichment for my-HSCs relative to bal-HSCs and HPCs (FIG. 1c-i). We developed independent antibody-conditioning regimens to deplete my-HSCs for each target, optimizing for multiple regulators of cell clearance, including: (i) anti-phagocytic signals, (ii) antibody density, and (iii) isotype.

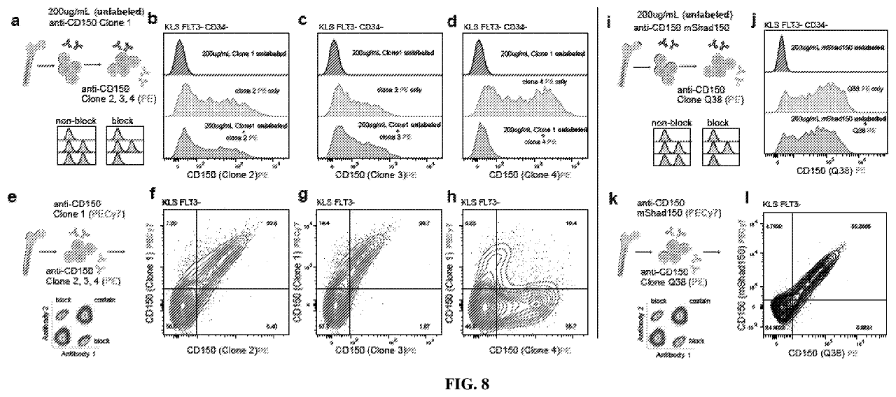

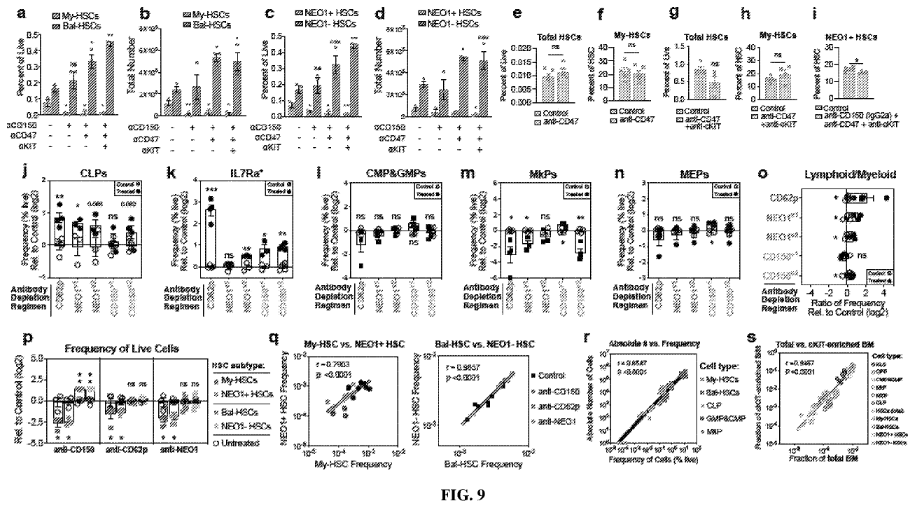

To determine the impact of targeting CD150, we tested anti-CD150 antibodies for their capacity to deplete my-HSCs in vivo. We administered rat IgG2b anti-CD150 antibodies to adult mice (6-7 months) and evaluated the bone marrow after approximately one week (FIG. 2b). To control for antibody masking, whereby in vivo treatment with anti-CD150 antibody might prevent the detection of target cells, we identified and validated independent non-masking antibodies to CD150 (FIG. 8a-l). Treatment of mice with anti-CD150 resulted in a significant depletion of my-HSCs relative to bal-HSCs by both frequency (FIG. 9a) and absolute number of cells (FIG. 9b and FIG. 9r). To verify the depletion of my-HSCs, we confirmed that HSCs expressing the independent my-HSC antigen NEO1 were also depleted with anti-CD150 (FIG. 9c). This finding and others described below rule out single antigen modulation as the mechanism for lack of CD150 in the HSC analyses, but favored depletion of cells with both markers. Consistent with rat IgG2b antibodies having greater depleting activity than IgG2a in mice, treatment with rat IgG2a anti-CD150 antibodies were only modestly effective (FIG. 9i). Collectively, these results demonstrated that antibody-targeting of CD150 was sufficient to deplete a significant fraction of my-HSCs in vivo.

We sought to further optimize the my-HSC depletion protocol by considering factors that limit in vivo cell clearance. Since antibody-mediated depletion can be limited by the anti-phagocytic signal CD47, we speculated that blocking CD47 could enhance the depletion of my-HSCs. Compared to anti-CD150 alone, dual treatment with anti-CD150 and anti-CD47 decreased the frequency of my-HSCs relative to bal-HSCs (FIG. 9a-b). To further decrease the threshold for cell-clearance, we added low-doses of anti-cKIT, which also depleted my-HSCs relative to bal-HSCs in the presence of anti-CD150 (FIG. 9a-b). Interestingly, the addition of anti-CD47 and/or anti-cKIT to anti-CD150 increased the frequency and absolute number of bal-HSCs in the bone-marrow after approximately one week (FIG. 9a-b), consistent with their expansion after my-HSC depletion. Overall, the most effective regimen to deplete my-HSCs relative to bal-HSCs was combining antibodies to CD150, CD47, and cKIT (FIG. 9a-b and FIG. 2a-c, 2f).

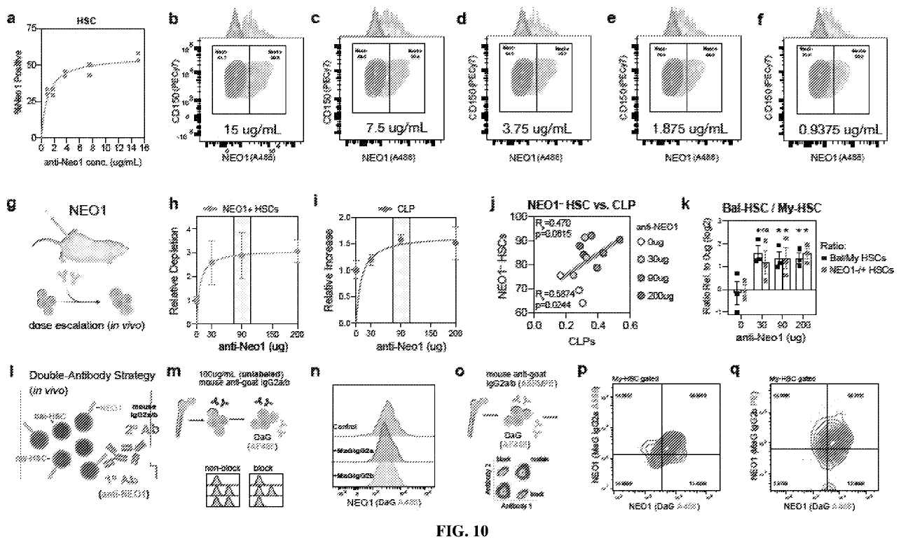

The optimized protocol we developed to deplete my-HSCs by targeting CD150 informed our strategy to deplete my-HSCs by targeting CD62p or NEO1. To target CD62p, we used an anti-CD62p antibody of mouse IgG2a isotype, which was predicted to have high depleting activity in mice. Indeed, administration of anti-CD62p in combination with anti-cKIT and anti-CD47 significantly depleted my-HSCs in the bone-marrow after approximately one week (FIG. 2d, 2g). To target NEO1, we combined goat anti-mouse NEO1 antisera with anti-CD47 and anti-cKIT, which also resulted in effective depletion of my-HSC in the bone marrow after approximately one week (FIGS. 2e, 2h and FIG. 10a-q). Similar to treatment with anti-CD150 (FIG. 9c-d), treatment with anti-CD62p or anti-NEO1 also depleted HSCs expressing the my-HSC antigen NEO1 (FIG. 9p-q). Taken together, these experiments established three separate antibody-conditioning regimens that deplete my-HSCs bearing two independent cell surface antigens in vivo. Again, depletion of cells marked by two independent surface antigens with antibodies to one marker only rules out antigenic modulation and favors my-HSC cellular depletions.

To verify changes in HSC composition after my-HSC depletion, we conducted gene expression profiling of purified total HSCs in aged (11 months) mice with or without antibody-conditioning (FIG. 2i). Gene Set Enrichment Analysis (GSEA) revealed that HSCs isolated from mice receiving antibody-conditioning were enriched in previously reported gene-signatures of young HSCs and bal-HSCs (FIG. 2j-k), and were depleted in gene-signatures of old HSCs and my-HSCs (FIG. 2j-k). Thus, in addition to depleting HSCs marked by validated my-HSC cell-surface proteins, antibody conditioning altered the molecular composition of the HSC compartment by selectively depleting the my-HSC RNA ‘fingerprint’ cells and retaining or expanding the young or bal-HSC RNA fingerprints.

Depletion of my-HSCs increases lymphoid progenitors and decreases myeloid progenitors. To determine the impact of depleting my-HSCs on downstream progenitors, we examined common lymphoid progenitors (CLPs) and myeloid progenitors (CMPs & GMPs, MkPs, and MEPs) in the bone marrow from mice receiving each antibody-conditioning regimen (FIG. 2a-b). After approximately one-week post-treatment, all three antibody conditioning protocols significantly increased the frequency of CLPs or IL7Ra⁺ lymphocyte precursors in the bone marrow (FIG. 2f-h and FIG. 9j-k). In addition, the protocols targeting CD62p or NEO1 decreased the frequency of myeloid progenitors (FIG. 2g-h and FIG. 9l-m) and increased the ratio of lymphoid progenitors (CLPs) to myeloid progenitors (CMPs & GMPs) by up to 4-fold (FIG. 9o). The increase in lymphoid progenitors and decrease in myeloid progenitors we observed upon my-HSC depletion in adult mice pointed to the potential of this treatment to reverse age-related immune decline.

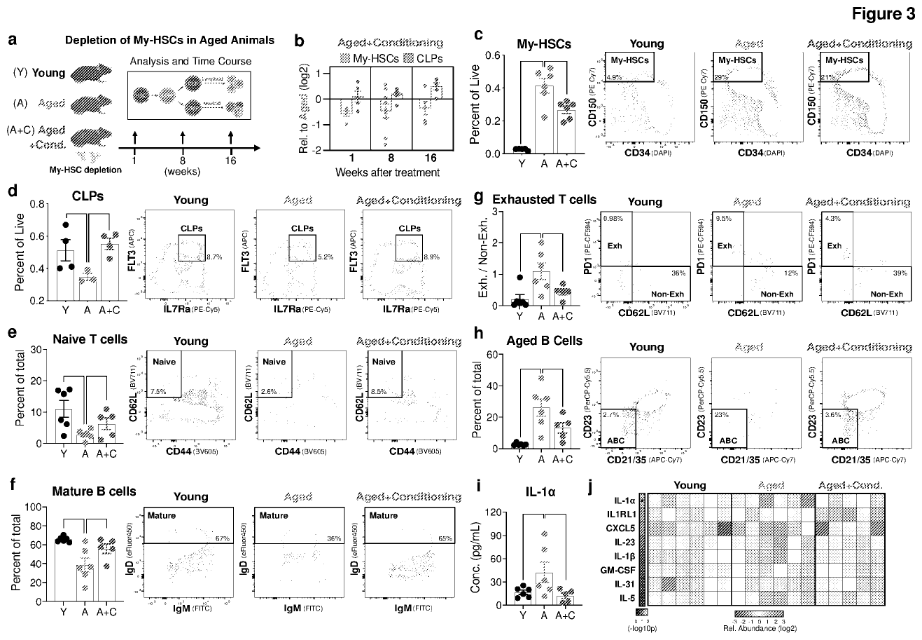

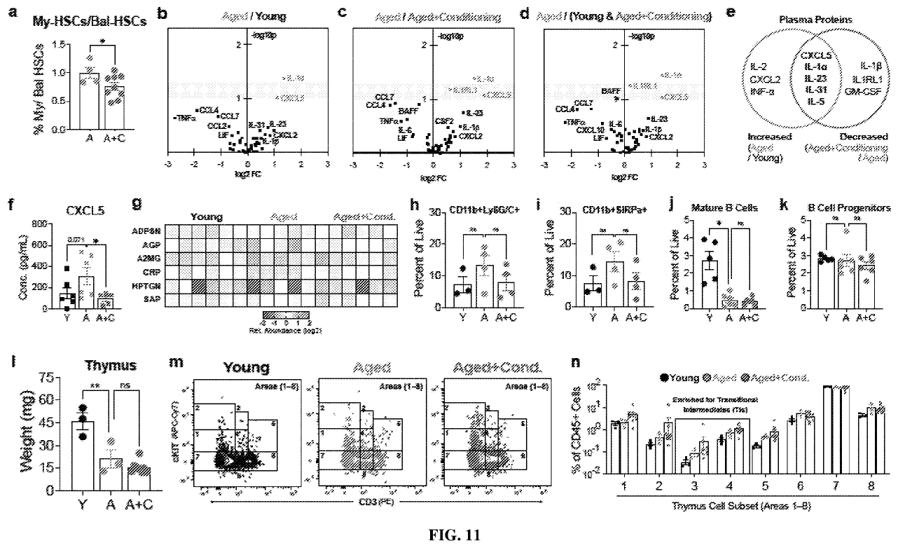

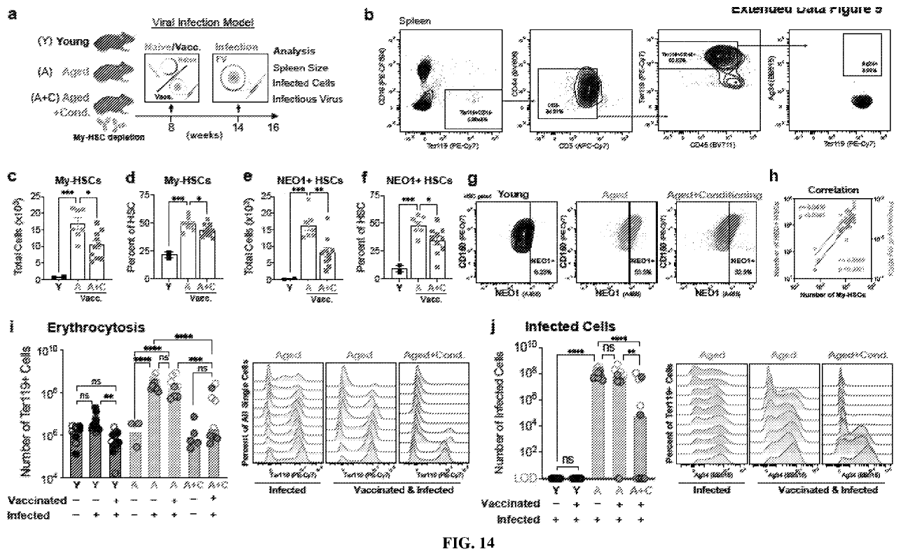

Depletion of my-HSCs in aged mice restores features of a youthful immune system. To determine if reversing the age-related shift from bal-HSCs to my-HSCs would restore cells critical for immune function and reverse age-related markers of immune decline, we compared young-adult mice (3-6 months) to aged mice (18-24 months) with or without anti-NEO1 antibody-conditioning (FIG. 3a). Phenotypic analyses were conducted after (i) approximately 1-week to evaluate acute effects, or (ii) approximately 8-weeks or 16-weeks to evaluate persistent effects, which is after the estimated clearance of antibodies and non-self-renewing cells (FIG. 3a). Aged mice receiving antibody-conditioning demonstrated a significant decrease in my-HSCs after approximately 1-week (FIG. 3b-c) with no significant acute impact on the frequency of mature B cells or myeloid cells (FIG. 11h-k). Interestingly, after approximately 8-weeks after treatment, the frequency of my-HSCs relative to bal-HSCs was significantly reduced (FIG. 11a), which we confirmed by absolute numbers of cells in the total bone-marrow in an independent experiment (FIG. 14c). Thus, antibody conditioning depleted my-HSCs in aged animals at least several months after a single administration. The impact on common lymphoid progenitors (CLPs), which are non-self-renewing progenitors, was evaluated next. Compared to young-adult mice, untreated aged mice demonstrated a significant decrease in the frequency of CLPs (FIG. 3d), but antibody-conditioned, aged mice showed increased frequencies of CLPs 8-weeks and 16-weeks after treatment (FIG. 3b, 3d). These results underscored the impact of a single administration of therapy to rejuvenate the hematopoietic stem cell and progenitor compartments.

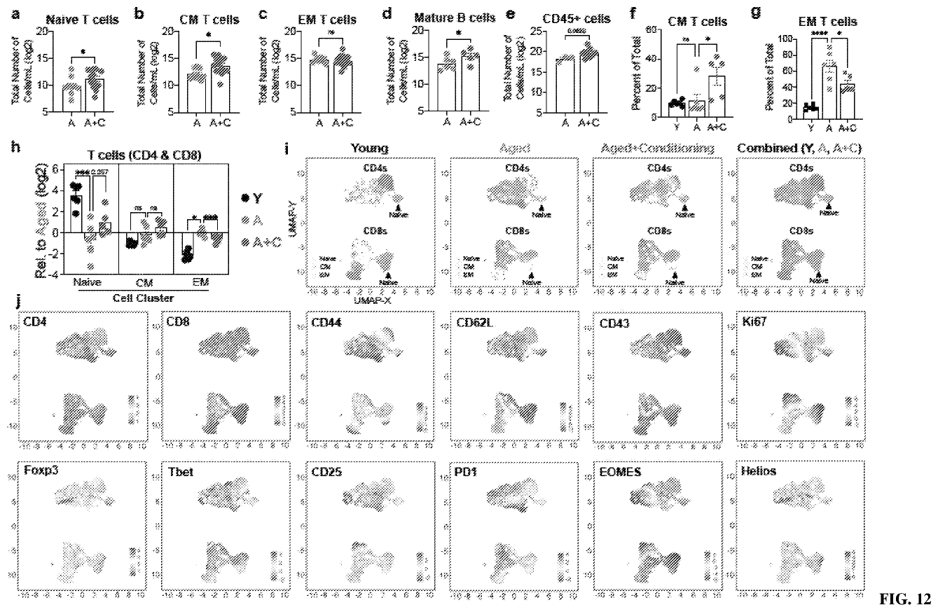

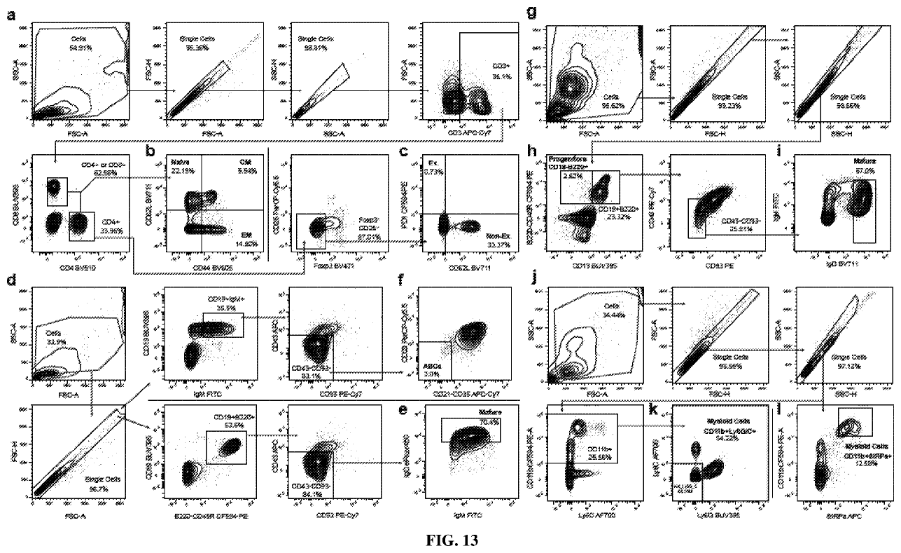

Antibody conditioning increases naïve T cells and B cells in aged mice. A critical deficit of aged immune systems is the reduced generation of T and B lymphocytes capable of recognizing novel antigens. Given that depletion of my-HSCs in aged mice increased lymphocyte progenitors, we sought to determine if these changes were sufficient to increase naïve T and B cells. We evaluated mice after 8-weeks, since the generation of new T and B cells from HSCs peaks between 7-11 weeks. Although we did not observe significant differences in thymus weight (FIG. 11l), treated mice contained all the thymic progenitor subsets associated with thymus function (FIG. 11m-n). After approximately 8-weeks, aged mice receiving antibody-conditioning demonstrated a significant increase in the frequency (FIG. 3e) and absolute number (FIG. 12a) of circulating naïve T cells (CD4+ or CD 8+, CD44⁻ CD62L⁺ cells) compared to age-matched controls. To further interrogate T cell subsets, we examined central (stem) memory (CD44⁺CD62L⁺) and effector memory (CD44³⁰ CD62L⁻) cells by canonical markers or by cluster-based analysis (FIG. 13a-b, 7f-j). Depletion of my-HSCs was associated with slightly increased central (stem) memory (CM) T cells by absolute number (FIG. 12 b), but the absolute number of EM (EM) T cells was not significantly impacted (FIG. 12c). Similar to our results for T cells, aged mice receiving antibody-conditioning also demonstrated a significant increase in the frequency (FIG. 3f) and the absolute numbers (FIG. 12d) of mature circulating B cells (CD19⁺B220⁺IgM⁺IgD⁺). Antibody treatment did not significantly impact the total number of circulating CD45⁺ leukocytes (FIG. 12e). Overall, these results demonstrated that antibody-mediated depletion of my-HSCs selectively increased both naïve T cells and mature B cells in aged mice.

Antibody conditioning decreases T and B cells with age-related cellular phenotypes. In addition to their decreased frequency and production in aged animals, lymphocytes undergo age-related accumulation of markers of exhaustion and/or inflammation that are thought to contribute to immune decline. In aged mice, CD4 T cells with an exhausted phenotype (PD1⁺CD62L⁻) increase relative to those with a non-exhausted phenotype (PD1⁻CD62L⁺), which we confirmed in our experimental cohort (FIG. 3g). Compared to aged controls, antibody-conditioning decreased exhausted T cells relative to non-exhausted T cells (FIG. 3g). Aged mice also accumulate a distinct population of ‘age-associated B cells’ (ABCs) correlated with reduced humoral immunity. Our control cohort of aged mice had an increased frequency of ABCs (CD19⁺IgM⁺CD93⁻CD43⁻CD21/CD35⁻CD23⁻) relative to young-adult mice, which was significantly decreased after antibody conditioning (FIG. 3h). Thus, in addition to increasing naïve T cells and mature B cells, antibody conditioning also suppressed lymphocyte age-related immunophenotypes. Collectively, these results suggested my-HSC depletion might enhance immune function in aged animals.

Antibody conditioning decreases systemic pro-inflammatory markers. In addition to immune cell phenotypes, aging is also associated with increased levels of circulating pro-inflammatory mediators, referred to as ‘inflammaging’, which has been linked to HSC dysfunction and myeloid bias. To determine if the depletion of my-HSCs in aged animals impacted pro-inflammatory mediators, the levels of a set of circulating proteins in plasma collected from young-adult and aged mice, with or without antibody conditioning, was evaluated after approximately 8-weeks. The most elevated proteins in aged animals relative to young-adult mice were the pro-inflammatory factors IL-1α and CXCL5 (FIG. 11b), which were also the most decreased proteins in aged mice receiving antibody-conditioning (FIG. 3i-j and FIG. 11c-d). Antibody-conditioning also decreased numerous additional pro-inflammatory mediators in aged mice, including IL-1β, CXCL2 (MIP-2), and IL-23 (FIG. 3j and FIG. 11c, 6e). Thus, in addition to resulting in a more youthful composition of immune cells, my-HSC depletion decreased the levels of circulating pro-inflammatory mediators several months after treatment.

Depletion of my-HSC enhances functional immunity to viral infection in aged mice. A hallmark of immune aging is reduced resistance to infection and responsiveness to vaccination, as became evident during the COVID-19 pandemic. To determine if my-HSC-depletion enhanced functional immunity to infection, we examined the vaccine-induced immune responses of mice to a pathogenic viral infection using the murine Friend retrovirus (FV) model. Vaccine-induced protection against FV involves a complex immune response that requires B cells and CD4+ and CD8+ T cells, each providing indispensable and non-redundant functions. The stringent immunological requirements for vaccine protection in the FV model provided a rigorous test for rejuvenation of immune responses in aged mice.

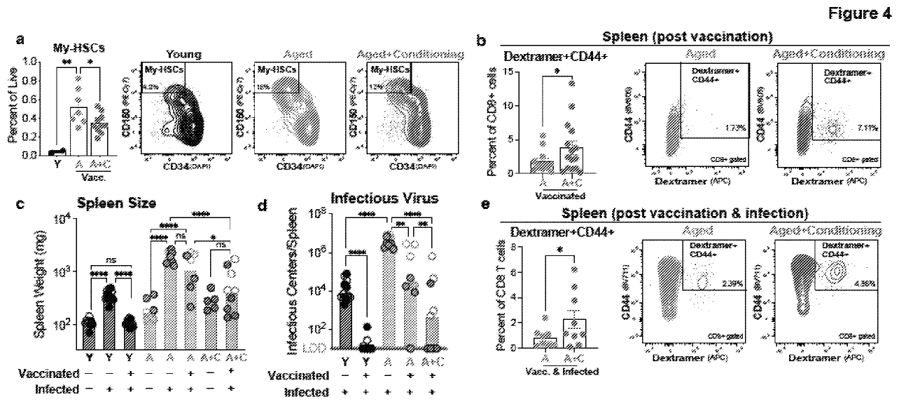

The generation of antigen specific antiviral CD8+ T cells is required for vaccine-induced immune protection from Friend virus. To evaluate the primary response to vaccination, aged mice were vaccinated intravenously (i.v.) with live-attenuated virus approximately 8-weeks after receiving anti-NEO1 antibody-conditioning. The spleens were harvested 10-14 days later at the peak of CD8+ T cell response. Aged mice receiving my-HSC depletion demonstrated an increase in virus-specific CD8+ T cell responses (dextramer+) following vaccination as compared to old mice (FIG. 4b), demonstrating that my-HSC depletion improved the response to vaccination. To evaluate functional vaccine-induced immune protection, aged mice were vaccinated approximately 8-weeks after receiving anti-NEO1 antibody-conditioning and then infected with FV 6-weeks after vaccination (FIG. 14a). Spleen cells were examined at two-weeks post-infection, the peak of virus replication. Controls included naïve mice, non-depleted aged mice, and unvaccinated mice. We verified that anti-NEO1 antibody conditioning decreased my-HSC by both frequency and absolute number under these conditions and time-points (FIG. 4a and FIG. 14c-h). Control young-adult mice inoculated with FV had approximately three-fold larger spleens than naïve mice (FIG. 4c) and a per spleen median of 7,000 infectious centers (FIG. 4d), a measure of live, infectious virus. Vaccination of young-adult mice prevented splenomegaly (FIG. 4c) and significantly reduced infectious centers (FIG. 4d). Aged-infected mice fared much worse than young-adult mice in all parameters associated with infection: their median increase in spleen weights was ten-fold (FIG. 4c) and their median infectious centers per spleen was 4 million (FIG. 4d), which was more than 500-fold the median in infected young-adult mice. Vaccination of aged mice resulted in a slight but not statistically significant decrease in splenomegaly (FIG. 4c), and only 1 out of 8 mice was able to control infection (FIG. 4d and FIG. 14). In contrast, mice that had been conditioned by my-HSC-depletion 2 months prior to vaccination demonstrated significantly reduced splenomegaly (FIG. 4c), and 4 of the 9 my-HSC-depleted mice had no detectable infectious centers in their spleens (FIG. 4d), the most stringent test of infection control. Consistent with the known role of antigen-specific CD8+ T cells in vaccine-induced FV protection, vaccinated aged mice receiving my-HSC-depletion had increased virus-specific CD8+ T cell responses (dextramer+) in the spleen compared to age-matched controls (FIG. 4e). Taken together, these results demonstrated that antibody-mediated depletion of my-HSCs in aged animals restored vaccine-induced immune function in response to infection with a live, pathogenic virus in vivo.

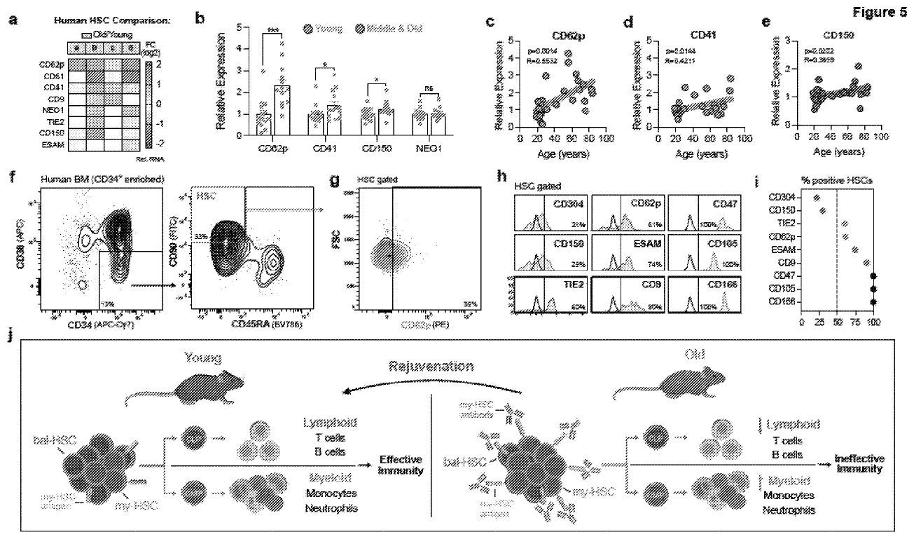

Mouse myeloid-biased HSC antigen targets are enriched in aged human HSCs. The age-associated expansion of HSCs with myeloid bias occurs in both mouse and humans. Having demonstrated that antibody-mediated depletion of my-HSCs reverses several features of age-related immune decline in mice, we investigated if the my-HSC antigenic targets used in our conditioning protocol might be applicable to humans. First, we sought to determine if human homologs to mouse my-HSC genes were expressed by aged human HSCs. Indeed, several mouse my-HSC genes, including CD62p (Selp), CD150 (Slamf1), and CD41 (Itga2b), were significantly increased in aged human HSCs from independent datasets (FIG. 5a-b and FIG. 60a-b) and were also correlated with age across adulthood (FIG. 5c-e and FIG. 60c-e). In addition, genes for multiple mouse my-HSC antigens were enriched in HSCs isolated from pathologies related to aging of the human hematopoietic system, including aberrant and pre-malignant human HSCs (FIG. 60f). Interestingly, the most enriched gene across all datasets—in both mouse and human—was CD62p. Thus, several genes for mouse my-HSC antigens were also enriched in human HSCs associated with age and disease.

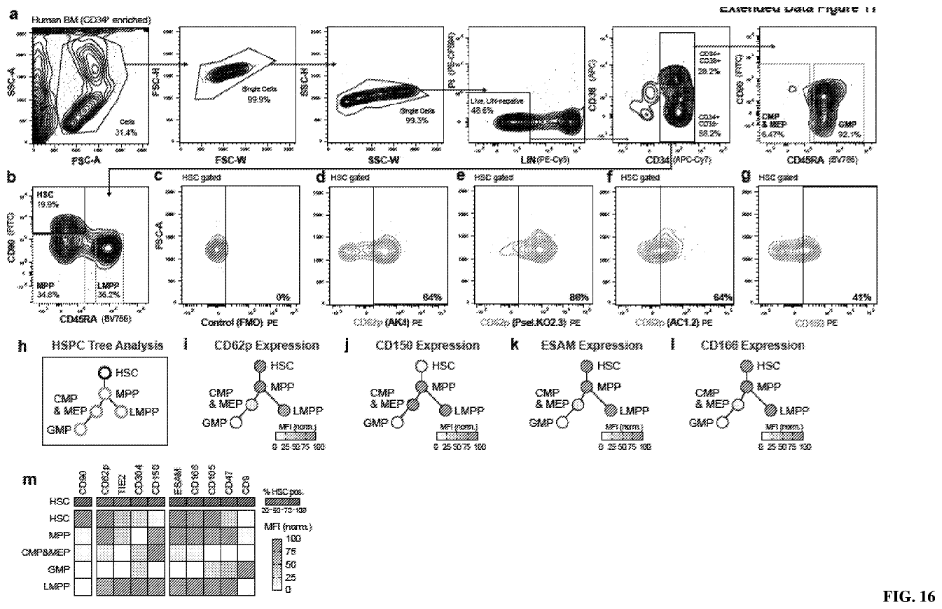

We next evaluated whether any of these candidate markers could be detected on human HSCs with antibodies. We examined the cell-surface protein expression of a subset of candidate antigens on highly pure populations of human HSCs (Lin⁻CD34⁺CD38⁻CD90⁺CD45RA⁻), which represent <10% of CD34⁺ cells in bone marrow (FIG. 61a-b). We previously demonstrated that antibodies to NEO1 marked a subpopulation of human HSCs. Antibodies to several additional cell-surface candidates—including CD62p and CD150—also separated human HSCs into two populations (FIG. 5f-i and FIG. 16c-g). These markers were expressed with varying degrees of enrichment on HSCs relative to downstream progenitors (FIG. 16h-m). Thus, all three mouse my-HSC markers that we demonstrated as therapeutic targets for my-HSC depletion in vivo—CD150, CD62p, and NEO1—were also present on the cell-surface of a subset of human HSCs. These experiments represent the first steps towards identifying therapeutic targets to deplete human my-HSCs and to rejuvenate the aged immune system with this strategy (FIG. 5j).

Aging is associated with an increase in myeloid-biased HSCs relative to balanced HSCs, which is thought to underlie several age-related immunological pathologies. We sought to develop a therapy to return the immune system to a more youthful state characterized by more HSCs with balanced production of lymphoid and myeloid lineage cells. We speculated that depletion of myeloid-biased HSCs would enable untargeted balanced HSCs to rejuvenate the immune system during aging. By identifying a core set of cell-surface antigens enriched in my-HSCs and using antibodies to these antigens (FIG. 1), we developed multiple conditioning protocols that deplete my-HSCs in vivo (FIG. 2). We demonstrated that depletion of my-HSCs relative to bal-HSCs restores several features of a youthful immune system, including increasing lymphocyte progenitors, naïve T cells, and B cells, while suppressing features of an aged immune system, including decreasing lymphocytes with dysfunctional markers, and decreasing inflammatory mediators (FIG. 3). Importantly, we demonstrate that depletion of my-HSCs through antibody-conditioning improves functional immunity in aged mice to live, pathogenic retroviral infection in vivo (FIG. 4). Finally, we uncovered that murine myeloid-biased HSC antigens also mark subsets human HSCs (FIG. 5), implicating them as candidate therapeutic targets to rejuvenate the blood-forming immune system in humans.

Etiology of hematopoietic stem cell clonal heterogeneity. Evolution of the vertebrate immune system occurred in the context of populations of individuals that were geographically limited. Immune responses to pathogens are predominated first by an innate response by cells of the myeloid lineage (macrophages, neutrophils, and granulocytes), and second by eliciting a more specific adaptive response by cells of the lymphoid lineage (B cells and T cells). Each of the millions of naïve B cells and T cells expresses a distinct receptor capable of recognizing a specific antigen from a pathogen, one receptor specificity for each antigen. Upon pathogen encounter, those cells with specificity expand into both effector cells to contain the pathogen, and into long-lived T and B stem/memory cells that can respond much faster and more potently if the pathogen is re-encountered. Before machine-mediated transportation—i.e., trains, planes, and cars—individuals were likely to be exposed to the majority of pathogens in their local geography by the time of reproductive age. Since T and B memory/stem cells can survive the lifetime of the individual, they should be sufficient to provide adaptive immune memory to all local microbial pathogens. Thus, the generation of new T and B lymphocytes in later life was likely no longer advantageous. In contrast, the production of short-lived myeloid cells would remain important for acute innate responses, even in later life.

In the aged individual, the requirement to maintain myeloid output for acute responses in the context of a sufficient long-lived T and B cell repertoire could explain the shifting of the HSC pool from balanced-HSCs to myeloid-biased HSCs. Although this biology has worked well throughout almost all of human evolution, the introduction of geographic travel and migration—by modern transportation including trains, planes, and cars—created novel exposures of individual to microbes and pathogens later in life when T and B cells are no longer efficiently produced. The shifting of the hematopoietic system to myeloid-biased production has likely enabled novel pathogens to cause global pandemics. As the COVID-19 pandemic caused by SARS-COV-2 has demonstrated, elderly patients are the most likely to die as a result of infection. Furthermore, annual influenza vaccinations often don't immunize the elderly, and often require much higher antigen doses, likely to stimulate the very few new lymphocytes produced from expanded my-HSC. Thus, even a transient reversal of my-HSC bias could enable bal-HSCs to produce a burst of new lymphocytes that could be protective and/or clinically effective against novel pathogens or during pandemics.

In addition to its impact on reducing adaptive immune responses, the expansion of myeloid-biased HSCs with age can contribute to aberrant immune inflammatory responses. The morbidity and mortality of elder patients infected with new respiratory pathogens such as new strains of influenza and SARS-COV-2 is not only because of poor and delayed adaptive immune response, but also because of dysfunctional inflammatory consequences. Myeloid-biased HSCs are pro-inflammatory, producing or eliciting inflammatory cytokines, which provide a much more serious response to microbes or endogenous antigens. Thus, the predominance of myeloid-biased HSCs in the elderly is a double-edged sword in the battle with novel pathogens, resulting not only in a poor adaptive immune response, but also in detrimental inflammatory responses. These heightened inflammatory responses driven by expansion of my-HSCs in the elderly may also contribute to chronic inflammatory disease, which can occur in the absence of a known pathogenic source. Rejuvenation of the blood-forming system through depletion of myeloid-biased HSCs can promote more functional immune responses by increasing the generation of new T and B cells and also by reducing the production of inflammatory myeloid cells.

Myeloid-biased hematopoietic stem cells in cancer. Targeting myeloid-biased hematopoietic stem cells may also have relevance to blood and solid cancers. Almost all cancers have an increased incidence as humans age. While this is in part due to the accumulation of driver mutations in pre-cancerous clones over long periods of time, the diminution of adaptive immunity and the confounding presence of a more inflammatory milieu in the aged likely contributes to an inability to recognize and eliminate newly arising cancers. Rejuvenation of the immune system with balanced HSCs could restore surveillance systems required for transformed and partially transformed cells that drive cancer and reduce the generation of myeloid cells that suppress tumor immunity. Such an approach could support T cell based immunotherapies such as immune-checkpoint blockade, or immunotherapies that would benefit from competent lymphocytes, such as cross-presentation due to macrophage-checkpoint blockade. Furthermore, this approach could reverse the inflammation and/or depletion of cells that result from cytotoxic anti-cancer therapies such as chemotherapy and radiotherapy.

Molecular regulators of HSC function in age and disease. The molecular mechanisms that regulate the expansion of myeloid-biased HSCs with age are not fully known. There are at least two models that may explain the expansion of myeloid-biased HSCs with age: (i) changes to the clonal competition between distinct subtypes of HSCs over time, or (ii) epigenetic changes to the functional properties of stem cells over time. This study is not intended to distinguish between these two models. Given the persistence of HSCs with balanced production of lymphoid and myeloid cell lineages in aged animals, our approach to rejuvenate the blood-forming system is independent of which model is correct.

Myeloid-biased hematopoietic stem cells and age-related immune decline. In this study, antibody-mediated depletion of my-HSCs enabled bal-HSCs to repopulate the HSC compartment, resulting not only reduced the inflammatory cytokines and exhaustion of adaptive immune responses associated with aging, but also promoted expansion of lymphoid cells including naïve T cells and mature B cells. Importantly, the experiments with pathogenic Friend retrovirus demonstrated that this rejuvenation of the immune system to the phenotype of a more youthful state allowed vaccine-induced immunity to control virus spread and pathology in aged mice that were otherwise not vaccine-protected. Since it has been shown that vaccine protection against FV requires non-overlapping and critical activity by B cells, CD4+ T cells and CD8+ T cells, the results indicate that the antibody conditioning protocol developed in this study induced broad rejuvenation of adaptive immunity. Such improvement in vaccine-induced protection against a live viral infection represents a significant advancement in reversing age-associated immune senescence. Furthermore, the persistent decrease in my-HSCs, increase in lymphocyte progenitor and naïve cells, and decrease in pro-inflammatory markers in old animals several months after antibody conditioning underscores the impact of a single administration of this treatment to rejuvenate the stem cell and progenitor compartments.

Myeloid-biased hematopoietic stem cells in age-related diseases. Aged humans not only have polyclonal contributions of HSCs to the blood system, but in many individuals, increase of HSC clones driven by loss of function of epigenetic modifiers that help open or close chromatin (e.g., TET2 and DNMT3A). While such clones exist in many otherwise normal individuals, as observed in clonal hematopoiesis of indeterminate potential (CHIP), they have a higher likelihood of progressing to MPN, MDS, and AML, as well as of developing atherosclerosis. If safe clinical protocols are developed to administer antibody cocktails such as those studied here, it is conceivable that amongst my-HSCs are those that are involved in CHIP and could progress to these myeloid diseases, AML, and the inflammations that occur in other age-related inflammatory and fibrotic conditions. Finally, aged humans have proven to be more susceptible to pathogenic viral infections such as influenza and COVID-19, both in becoming infected and in progressing to morbidity and mortality more often than young humans. Our study provides proof-of-principle for future translational studies focused on applying similar antibody-conditioning strategies to improve functional immunity combat infections, chronic disease, and cancer in humans.

Rejuvenation of the human immune system. This study demonstrates that rejuvenation of at least some aspects of the HSC derived hematolymphoid system is possible by antibody treatments without cytotoxic chemotherapy or radiotherapy. The conservation between mouse and humans of the expansion of myeloid-biased HSCs and of the genes that increase during HSC aging show that this pre-clinical study supports the development of clinical therapies to rejuvenate the blood-forming system in patients. These mouse studies herein point the direction of which markers on human my-HSC are effective or reasonable targets.

### Methods

Animal Experiments. All mice were C57BL/6 or (C57BL/10×A.BY)F1 (H-2^{b/b}, Fv1^{b}, Rfv3^{r/s}) and between 8-weeks to 120-weeks old. Mouse ages were defined as follows: mature young-adult (3 to 6 months; 12 to 24 weeks), middle-aged (10 to 14 months; 40 to 56 weeks), and aged (18 to >24 months; 72 to >96 weeks). For the young vs. aged time-course experiment, mature young-adult (3 to 6 months) and aged (18 to >24 months) mice were compared. For identification and validation of my-HSC markers, mice 6-12 months were used (e.g., between mature young-adult and middle-aged). For routine antibody validation experiments, mature young-adult (3 to 6 months) mice were used. Mice were routinely monitored, and abnormal or sick mice were excluded from further analysis. Mice were bred and maintained at Stanford University's Research Animal Facility or at the Rocky Mountain Laboratories. All animal experiments were performed according to guidelines established by the Administrative Panel on Laboratory Animal Care of Stanford University or on an Animal Study Proposal approved by the Animal Care and Use Committee of the Rocky Mountain Laboratories (RML 2018-058, RML 2021-046) and carried out by certified staff in an Association for Assessment and Accreditation of Laboratory Animal Care International-accredited facility according to the institution's guidelines for animal use, the basic principles in the NIH Guide for the Care and Use of Laboratory Animals, the Animal Welfare Act, and the United States Department of Agriculture and the United States Public Health Service Policy on Humane Care and Use of Laboratory Animals.

Bone Marrow Cell Isolation. Mice were euthanized and bone marrow was harvested following one of two methods. The unilateral or bilateral femurs, tibias, and pelvises were dissected, cleaned, and collected in a mortar bowl containing PBS supplemented with 2% FBS (FACS-buffer) and 1 mg/mL DNAse-I (LS002007; Worthington). Bones were crushed, and the resulting cell suspension was passed through a 40 μm filter. Alternatively, the femurs and tibias were dissected, cleaned, and cut at the joints and the bone marrow was flushed using an inserted 25-gauge needle and phosphate-buffered balanced salt solution (PBBS) with cells passed through a 100 μm filter. Cells were collected by centrifugation and washed with FACS-buffer multiple times. Red blood cells were depleted by ACK-lysis or by cKIT-enrichment. For ACK-lysis, cells were resuspended in 1 mL ACK Lysing Buffer (A1049201; ThermoFisher) and incubated for 10 minutes at room-temperature. For cKIT-enrichment, cells were Fc-blocked by incubation with 1 mg/mL rat IgG (ab37361; abcam) for 30 minutes on ice, followed by the addition of anti-cKIT APC-eFluor 780(47 -1171-82; ThermoFisher) for 30 minutes. Cells were collected by centrifugation and resuspended in FACS-buffer containing 10 μL anti-APC MicroBeads (130-090-855; Miltenyi Biotec) and incubated for 20 minutes on ice. Cells were then washed and isolated with LS Columns (130-042-401; Miltenyi Biotec) using a MACS Separator (Miltenyi Biotec) according to manufacturer instructions.

Flow cytometry. Flow cytometry was performed on a FACS Aria II (BD Biosciences) or FACS Symphony (BD Biosciences). For absolute cell counts, cells were counted prior to flow-cytometry, or a known volume of Precision Count Beads™ (424902; BioLegend) was added to a known volume of cells, and calculations were performed according to manufacturer's instructions. For all experiments with Precision Count Beads™, the stock concentration was assumed to be 1×10⁶ particles/mL, based on manufacturer's documentation. For mouse flow-cytometry, immunophenotypic analysis was performed on whole-bone marrow or on cKIT-enriched cells, isolated as described above. Prior to antibody staining, cells were Fc-blocked by incubation with 1 mg/mL rat IgG (ab 37361; abcam) for 30 minutes on ice. Antibody staining was performed in FACS-buffer solution (PBS with 2% FBS and DNAse-I). Incubations were performed on ice for at least 30 min. For HSC and progenitor analysis, cells were stained with combinations of the following antibodies (hereafter: HSPC stain): anti-FLT3 APC (ThermoFisher; 17-1351-82) or PerCP-eFluor 710 (eBioscience; 46-1351-82), goat anti-mouse NEO1 (R&D; AF1079), anti-CD150 PE-Cy7 (BioLegend; 115914; clone TC 15-12F12.2), anti-IL7Ra PE-Cy5 (ThermoFisher; 15-1271-82 or BioLegend; 135016) or APC (BioLegend; 135012), anti-CD16/32 BV510 (BioLegend; 101308), anti-cKit APC-eFluor780 (ThermoFischer; 47-1171-82), anti-mouse Lineage Cocktail (includes anti-CD3, anti-Ly-6G/C, anti-CD11b, anti-CD45R, anti-Ter-119) AF700 (BioLegend; 133313), anti-CD48 BV711 (BD; 740687), anti-CD41 BV650 (BD; 740504), anti-CD34 biotin (ThermoFisher; 13-0341-85), SCA1 BUV395 (BD; 744328), followed by Streptavidin BUV737 (BD; 612775) and donkey anti-goat IgG H&L AF488 (abcam; ab150129). In some instances, anti-CD150 clone mShad150 PE (eBioscience; 12-1502-80) or PE-Cy7 (eBioscience; 25-1502-82), anti-CD150 clone 9D1 PE (eBioscience; 12-1501-80), anti-CD150 clone Q38-480 PE (BD; 562651), anti-CD62 p PE (BioLegend; 148308), or anti-Ly6D PE (eBioscience; 12-5974-80), were included. For testing of candidate my-HSC markers, the following antibodies were used: anti-CD51 PE (12-0512-81; ThermoFisher), anti-CD61 PE (561910; BD), anti-CD31 PE (561073; BD), anti-CD38 PE (12-0381-81; ThermoFisher), anti-CD47 clone MIAP301 PE (127507; BioLegend), anti-CD47 clone MIAP410 PE (LS-C810701-25; LSBio), anti-CD62p PE (148305; BioLegend), anti-ALCAM PE (12-1661-82; ThermoFisher), anti-CD9 PE (124805; BioLegend), anti-ESAM PE (136203, BioLegend), anti-TIE2 PE (124007; BioLegend), anti-CD201 PE (141503; BioLegend), or anti-cKIT clone ACK2 PE (135105; BioLegend). To calculate the absolute number of HPCs (e.g., CMP&GMP, MkP, MEP, CLP, etc.) the absolute numbers of cells was quantified in total bone marrow (non-cKIT enriched). To quantify the absolute number of HSCs and HSC subsets (e.g., my-HSCs, bal-HSCs, etc.), the absolute numbers of cells was quantified in total bone marrow (non-cKIT enriched), or the percentage of HSC/HSC subsets per KLS (Lin⁻cKIT⁺Sca1⁺) cells was calculated in the cKIT-enriched fraction and multiplied by the total number of KLS cells quantified in a paired sample of total bone marrow (non-cKIT enriched).

For T cell analysis, cells were stained with the following antibodies: anti-Helios AF647 (BD; 563951), anti-CD3 APC-Cy7 (BioLegend; 100222), anti-Ki67 R718 (BD; 566963), anti-CD43 AF488 (BioLegend; 121210), anti-CD8 BUV395 (BD; 563786), anti-Foxp3 eF450 (Invitrogen; 48-5773-82), anti-CD4 BV510 (BioLegend; 100559), anti-CD44 BV605 (BD; 563058), anti-CD62L BV711 (BioLegend; 104445), anti-EOMES PE (Invitrogen; 12-4875-82), anti-PD1 PE-CF594 (BD; 562523), anti-Tbet PE-Cy7 (Invitrogen; 25-5825-82), anti-CD25 PerCP-Cy5.5 (BioLegend; 102030). FV-specific CD8⁺ T cells were identified using H-2D^{b}/Abu-Abu-L-Abu-LTVFL APC-or PE-D^{b}gagL-MHC Dextramer (Immudex, Copenhagen, Denmark) at 1:25 during surface staining. For B cells analysis, cells were stained with the following antibodies: anti-CD43 APC (BioLegend; 121214), anti-CD21/CD35 APC-Cy7 (BioLegend; 123418), anti-CD5 AF700 (BioLegend; 100636), anti-IgM FITC (Invitrogen; 11-5790-81), anti-CD19 BUV395 (BD; 563557), anti-IgD eFluor450 (eBioscience; 48-5993-82), anti-CD11b BV510 (BioLegend; 101245), anti-MHCII BV605 (BD; 563413), anti-CD40 BV711 (BD; 740700), anti-PDL1 PE (Invitrogen; 12-5982-82), anti-CD93 PE-Cy7 (BioLegend; 136506), anti-CD23 PerCP-Cy5.5 (BioLegend; 101618), and anti-CD45R/B220 PE-CF594 (BD; 562290). Intracellular staining was performed as described. For erythroid cell analysis, spleen cells were first incubated for 30 min with mAb 34, a mouse IgG2b specific for the FV glycoGag protein expressed on infected cells, then stained with anti-mouse IgG2b FITC (BD; 553395) and anti-Ter119 PE-Cy 7 (Invitrogen; 25-5921-82). Cells from uninfected controls were used for gating strategy. For non-fixed cells, to determine viability, cells were incubated in buffer containing SYTOX Red Dead Cell Stain (Life Technologies) or SYTOX Blue Dead Cell Stain (ThermoFisher; S34857).

For flow-cytometry computational analysis, samples were first analyzed in FlowJo v10 Software (BD Life Sciences) and the T cell (CD4+ and CD8+) gated events were down-sampled to an equivalent number of cells per condition (Young, Aged, Aged+Conditioning), and the channel data for each sample was exported as CSV files. The Spectre package was applied to data from all samples using R (version 4.2.2), which were annotated and merged, and clusters were assigned with PhenoGraph, followed by dimensionality reduction with Uniform Manifold Approximation and Projection (UMAP) for visualization. Cluster assignments were annotated and/or merged based on prior knowledge of phenotypes for naïve, central memory (CM), and effector memory (EM) T cell subtypes.

To identify anti-CD150 antibodies that are not blocked by anti-CD150 antibody clone 1 (TC15-12F12.2, TC15), bone-marrow HSPC stained cells were incubated with saturating concentrations (200 ug/mL) of unlabeled anti-CD150 antibody clone TC15 and then stained with PE-conjugated anti-CD150 clones 2 (Q38), 3 (9D1), or 4 (mShad150); PE-Cy7 conjugated anti-CD150 clone TC15 was used as a control. To confirm if any anti-CD150 clones identify the same population of cells as anti-CD150 antibody clone 1 (TC15) by flow-cytometry, bone-marrow HSPC stained cells were incubated with PECy-7 anti-CD150 antibody clone 1 (TC15) and with either PE-conjugated anti-CD150 clone 2 (Q38), 3 (9D1), or 4 (mShad150). To confirm that anti-CD150 antibody clone 4 (mShad150) does not block anti-CD150 clone 2 (Q38), bone-marrow cells were incubated with saturating concentrations (200 ug/mL) of unlabeled anti-CD150 clone mShad150 and then stained with PE-conjugated anti-CD150 clone Q38; PE-Cy7 conjugated anti-CD150 clone mShad150 was used as a control. To confirm that anti-CD150 clone mShad150 and clone Q38 identify the same populations by flow-cytometry, bone-marrow cells were incubated with PECy-7 anti-CD150 clone mShad150 and with PE anti-CD150 clone 2 (Q38).

To confirm that mouse IgG2a (SB115d; SouthernBiotech) and IgG2b (SB115h; SouthernBiotech) anti-goat antibodies do not block donkey anti-goat IgG AF488 (abcam; ab150129), bone-marrow HSPC stained cells were incubated with saturating concentrations (100 ug/mL) of unlabeled mouse IgG2a (6158-01; SouthernBiotech) or IgG2b (6157-01; SouthernBiotech) anti-goat antibodies and then stained with donkey anti-goat AF488. To confirm that mouse IgG2a and IgG2b anti-goat antibodies identify the same populations as donkey anti-goat IgG AF488 by flow-cytometry, bone-marrow HSPC stained cells were incubated with mouse IgG2a AF555 (6158-32; SouthernBiotech) or IgG2b PE (6157-09; SouthernBiotech) anti-goat antibodies, and with donkey anti-goat AF488.

For human flow-cytometry, bone marrow mononuclear cells from young-adult donors (ages 26-33) were commercially obtained (AllCells, Inc.). CD34-positive cells were enriched with CD34 MicroBead Kit (130-046-702; Miltenyi Biotec) according to manufacturer instructions. Antibody staining was performed in FACS-buffer solution (PBS with 2% FBS and DNAse-I) at a 1:1 ratio to Brilliant Stain Buffer (563794; BD Biosciences). Non-specific binding was blocked with FcR Blocking Reagent (130-059-901; Miltenyi Biotec) for 5 minutes on ice, followed by the addition of the following antibodies: anti-lineage panel PE-Cy5 (anti-CD3, anti-CD4, anti-CD8, anti-CD11b, anti-CD14, anti-CD19, anti-CD20, anti-CD56, anti-CD235a), anti-CD34 APC-Cy7 (343514; Biolegend), anti-CD45RA BV-785 (304139; Biolegend), anti-CD38 APC (555462; BD), anti-CD90 FITC (328107; Biolegend), and one of anti-human PE: anti-CD62P clone AK4 (304905; Biolegend), anti-CD62P clone Psel. KO2.3 (12-0626-82; eBioscience), anti-CD62P clone AC1.2 (550561; BD), anti-CD150 (306307; Biolegend), anti-TIE2 (CD202b, 334205; Biolegend), anti-ESAM (408519; Novus), anti-CD166 (ALCAM, 343903; Biolegend), anti-CD9 (312105; Biolegend), anti-CD105 (Endoglin, 800503; Biolegend), or anti-CD304 (Neuropilin-1, 354503; Biolegend). All flow cytometry data were analyzed with FlowJo v10 Software (BD Life Sciences).

Antibody Conditioning. For antibody conditioning experiments, mice received injections of antibodies resuspended in PBS intraperitonially, unless otherwise specified. Control animals received an equivalent volume of PBS or an equivalent amount of isotype control antibodies: mouse IgG1 (clone MOPC-21, Bio X Cell), rat IgG2b (clone LTF-2, Bio X Cell), or rat IgG2a (clone RTK2758, BioLegend). Given that isotype control antibodies demonstrated no impact on phenotype, PBS was used as a control in many experiments to minimize costs, as described. My-HSC specific antibodies (anti-CD150, anti-CD62p, or anti-NEO1) were injected on Day-9. For CD150, 200 μg rat IgG 2b anti-CD150 (clone mShad 150, eBioscience) for CD150^{v1} protocol, or 200 μg rat IgG2a anti-CD150 (clone TC15-12F12.2, BioLegend) for CD150^{v2} protocol, was as administered on Day −9. For CD62p, 200 μg mouse anti-CD62p (clone RMP-1, BioLegend) was administered on Day −9. For NEO1, 30 μg, 90 μg, or 200 μg goat anti-NEO1 (polyclonal cat #AF1079, R&D) was administered on Day −9 for NEO1^{v1} protocol, and when indicated, 150 μg mouse IgG2a (SB115d; SouthernBiotech) or IgG2b (SB115 h; SouthernBiotech) anti-goat was administered 24-hours later on Day −8, for NEO1^{v2} protocol. For CD47-blockade, mouse IgG1 anti-CD47 (clone MIAP410, Bio X Cell) was administered on Day −11 (100 μg) and on Days −9 to Day −5 (500 μg daily), as previously described. For cKIT, rat anti-cKIT (clone ACK2, Bio X Cell) was injected retro-orbitally on Day −9 (30 μg, 50 μg, or 100 μg), and mice were administered 400 μg of diphenhydramine at least 30 min prior to administration, as previously described. Mice were euthanized for bone-marrow analysis on Day 0 (e.g., approximately 1-week), at approximately 8-10 weeks, or at approximately 14-16 weeks.

Blood Cell Isolation and Plasma Immunoassays. For blood cell isolation and plasma immunoassays, mouse peripheral blood was collected in EDTA tubes after removal of cells through centrifugation at 500 RCF for 10 min, whereupon plasma was transferred to a clean tube and centrifuged for an additional 10 min at 13,000 RCF, while the red blood cells were depleted with ACK-lysis, followed by a PBS wash, and then stained for flow cytometry as described above. For absolute cell counts per mL, the volume of blood obtained per animal was recorded, and a known volume of Precision Count Beads™ (424902; BioLegend) was added to a known volume of cells, and calculations were performed according to manufacturer's instructions assuming a Precision Count Beads™ stock concentration of 1×10⁶ particles/mL. Plasma was frozen at −80 C until processing by the Stanford Human Immune Monitoring Center (HIMC), as described. Samples were run in technical triplicate using the 48-Plex Mouse ProcartaPlexPanel™ (EPX480-20834-901; ThermoFisher Scientific) or the Mouse Acute Phase Magnetic Bead Panel 2 (MAP2MAG-76K; Millipore Sigma). MFI average value were compared after removal of statistical outliers using the extreme studentized deviate (ESD) Grubbs statistical test (a=0.0001). For comparison of estimated concentrations, values below the limit of detection were assigned the value equal to this lower limit.

Friend Virus Mouse Model. Ethics and biosafety statement. All in vivo experiments were performed in accordance with Animal Study Proposal approved by the Animal Care and Use Committee of the Rocky Mountain Laboratories (RML 2018-058, RML 2021-046) approved by the Institutional Animal Care and Use Committee of Rocky Mountain Laboratories (National Institutes of Health [NIH]) and carried out by certified staff in an Association for Assessment and Accreditation of Laboratory Animal Care International-accredited facility, according to the institution's guidelines for animal use, following the guidelines and basic principles in the NIH Guide for the Care and Use of Laboratory Animals, the Animal Welfare Act, and the United States Department of Agriculture and the United States Public Health Service Policy on Humane Care and Use of Laboratory Animals.

Vaccination and virus challenge. The Friend retrovirus (FV) stock used in these experiments was FV-NB, a lactate dehydrogenase virus (LDV)-free complex containing NB-tropic Friend murine leukemia helper virus (F-MuLV) and polycythemia-inducing spleen focus-forming virus (SFFV) generated as a spleen cell homogenate from infected BALB/C mice. The live attenuated vaccine was an NB-tropic F-MuLV helper stock, which replicates poorly without SFFV-induced proliferation, generated as a supernatant from infected Mus dunni cells. Mice of (C57BL/10×A.BY)F1 background were vaccinated by 0.1 ml intravenous (i.v.) injection of 10⁵ focus-forming units (FFU) of virus in phosphate-buffered, balanced salt solution (PBBS). For challenge, mice were injected i.v. with 0.2 ml PBBS containing 20,000 spleen focus-forming units of FV-NB complex.

Infectious centers assay. Titrations of single cell spleen suspensions were plated onto susceptible Mus dunni cells and allowed to incubate in vitro for 2 days at 37 °C and 5% CO2. The plates were then fixed with 95% ethanol, stained with F-MuLV envelope-specific Mab 72015219, followed by goat anti-mouse (H+L) HRP (EMD Millipore; AP308P), and then developed with aminoethylcarbazol substrate to detect foci.

Antigen-expressing cells in vivo. To quantify Ag34+ expressing cells in vivo, Ag34 expression was determined by mAb 34 antibody staining by flow-cytometry. Cells from uninfected controls were used to define the background level of staining. A positive vs. negative threshold was set equal to the highest level of background staining observed in non-infected animals, and only samples with values higher than this threshold were considered positive. Samples with values equal to or lower than background level in non-infected animals were considered negative and their level of staining was set to a value of 0%. Both positive and negative samples were included in the statistical analysis for comparison. To obtain the absolute number of Ag34+Ter119+ cells per samples, an equivalent number of cells were analyzed for each mouse by flow-cytometry, and the frequency of Ag34+Ter119+ cells (as defined by the positive and negative thresholds) per total cells was multiplied by the total number of cells counted per spleen.

Transcriptomic Analysis. Murine and Human HSCs. The following datasets were used to compare mouse old vs. young HSCs: Beerman (a, GSE43729), Bersenev (b, GSE39553), Flach (c, GSE48893), Maryanovich (d, GSE109546), Norddahl (e, GSE27686), Wahlestedt (f, GSE44923), Renders (g, GSE128050), Sun (h, GSE47819). The following datasets were used to compare mouse myeloid-biased HSCs vs. balanced HSCs: Gulati (i, GSE130504), Montecino-Rodriguez (j, GSE112769) Sanjuan-Pla (k, E-MEXP-3935). The following datasets were used to compare human old vs. young HSCs: Pang (a, GSE32719), Adelman (b, GSE104406), Nilsson (c, GSE69408), Hennrich (d, GSE115348). Additional transcriptional datasets related to human HSCs included: Kumar (e, HMGA2⁺ vs. HMGA2⁻ CD34⁺ cells, GSE107594), Tong (f, Essential Thrombocythemia & Polycythemia Vera vs. Normal HSCs, GSE111410), Woll (g, MDS vs. Normal HSCs, GSE55689), Corces (e, Pre-Leukemic vs. Normal HSCs, GSE74246). Data was processed and analyzed with GREIN or GEO2R.

Murine progenitors, mature cells, and tissues. To determine gene expression of mouse progenitors and mature cells, processed data was obtained directly from Gulati on 23 hematopoietic phenotypes based on 64 microarray expression profiles extracted by the Gene Expression Commons. Gene expression data from bulk mouse tissues was obtained from: Tabula Muris (GSE132040) and (Kadoki, GSE87633). Data was processed with Phantasus (v1.19.3).

RNA-sequencing of FACS-purified mouse HSCs. For RNA-sequencing of purified mouse HSCs, approximately 1,000 total HSCs (KLS FLT3⁻CD34⁻CD150⁺) were FACS-sorted from aged control mice or aged mice that received antibody-conditioning 9 days earlier and immediately added to lysis buffer. Libraries were prepared using Takara SMART-Seq v4 Ultra low Input RNA kit and sequencing was performed with NovaSeq with approximately 20 million paired reads per sample by MedGenome Inc. Differential gene expression was performed using DESeq2 with fold change shrinkage. Heatmaps were generated using Phantasus (v1.21.5) with FPKM values as input and Limma to define differentially expressed genes. GSEA was conducted on genes ranked by DESeq2 test statistic using WEB-based GEne SeT Analysis Toolkit (WebGestalt 2019) with default parameters using a custom list of curated gene-signatures. The following datasets were used to obtain gene-signatures Young vs. Old HSCs: Svendsen (i), Kuribayashi (ii), Maryanovich (iii, GSE109546), Norddahl (iv, GSE27686), Montecino-Rodriguez (v, GSE112769), Wahlestedt (vi, GSE44923), Mann (vii, GSE100428), Renders (viii, GSE128050). The following datasets were used to obtain gene-signatures of mouse myeloid-biased (my-) HSCs vs. balanced (bal-) HSCs: Mann (i, GSE100428), Montecino-Rodriguez (ii, GSE112769), Gulati (iii, GSE130504). Gene-signatures were obtained directly from these studies or were generated by identifying statistically significant differentially expressed genes between cell populations. Data was processed and analyzed with GREIN or GEO2R.

Statistical Analysis. All statistical analysis was performed using GraphPad Prism (GraphPad Software) or SPSS Statistics (IBM), unless otherwise specified. The statistical tests used (parametric or non-parametric t-test, one-way ANOVA, Pearson or Spearman correlation) and details (sample size, one-or two-tailed, multiple comparison adjustment) are noted in the figure legends. Throughout figures, error bars denote mean +/−SEM, unless otherwise specified. For p-values, * is <0.05, ** is <0.005, *** is <0.0005.

### REFERENCES

Adelman, E. R., Huang, H. T., Roisman, A., Olsson, A., Colaprico, A., Qin, T., Lindsley, R. C., Bejar, R., Salomonis, N., Grimes, H. L., and Figueroa, M. E. (2019). Aging Human Hematopoietic Stem Cells Manifest Profound Epigenetic Reprogramming of Enhancers That May Predispose to Leukemia. Cancer Discov 9, 1080-1101.10.1158/2159-8290.CD-18-1474.
Akashi, K., Traver, D., Miyamoto, T., and Weissman, I. L. (2000a). A clonogenic common myeloid progenitor that gives rise to all myeloid lineages. Nature 404, 193-197.
Akashi, K., Traver, D., Miyamoto, T., and Weissman, I. L. (2000b). A clonogenic common myeloid progenitor that gives rise to all myeloid lineages. Nature 404, 193-197.
Baum, C. M., Weissman, I. L., Tsukamoto, A. S., Buckle, A. M., and Peault, B. (1992). Isolation of a candidate human hematopoietic stem-cell population. Proc Natl Acad Sci USA 89, 2804-2808.
Beerman, I., Bhattacharya, D., Zandi, S., Sigvardsson, M., Weissman, I. L., Bryder, D., and Rossi, D. J. (2010). Functionally distinct hematopoietic stem cells modulate hematopoietic lineage potential during aging by a mechanism of clonal expansion. Proc Natl Acad Sci USA 107, 5465-5470.10.1073/pnas.1000834107.
Beerman, I., Bock, C., Brian, Zachary, Gu, H., Meissner, A., and Derrick (2013). Proliferation-Dependent Alterations of the DNA Methylation Landscape Underlie Hematopoietic Stem Cell Aging. Cell Stem Cell 12, 413-425.10.1016/j.stem.2013.01.017.
Bersenev, A., Rozenova, K., Balcerek, J., Jiang, J., Wu, C., and Tong, W. (2012). Lnk deficiency partially mitigates hematopoietic stem cell aging. Aging Cell 11, 949-959. 10.1111/j.1474-9726.2012.00862.x.
Bhattacharya, D., Cheah, M. T., Franco, C. B., Hosen, N., Pin, C. L., Sha, W. C., and Weissman (2007). Transcriptional profiling of antigen-dependent murine B cell differentiation and memory formation. J Immunol 179, 6808-6819.
Boivin, G., Faget, J., Ancey, P. B., Gkasti, A., Mussard, J., Engblom, C., Pfirschke, C., Contat, C., Pascual, J., Vazquez, J., et al. (2020). Durable and controlled depletion of neutrophils in mice. Nat Commun 11, 2762. 10.1038/s41467-020-16596-9.
Bournazos, S., Gupta, A., and Ravetch, J. V. (2020). The role of IgG Fc receptors in antibody-dependent enhancement. Nature Reviews Immunology 20, 633-643. 10.1038/s41577-020-00410-0.
Challen, G. A., Boles, N. C., Chambers, S. M., and Goodell, M. A. (2010). Distinct Hematopoietic Stem Cell Subtypes Are Differentially Regulated by TGF-β1. Cell Stem Cell 6, 265-278.
Chao, M. P., Alizadeh, A. A., Tang, C., Myklebust, J. H., Varghese, B., Gill, S., Jan, M., Cha, A. C., Chan, C. K., Tan, B. T., et al. (2010). Anti-CD47 antibody synergizes with rituximab to promote phagocytosis and eradicate non-Hodgkin lymphoma. Cell 142, 699-713.
Chen, C., Liu, Y., and Zheng, P. (2009). mTOR regulation and therapeutic rejuvenation of aging hematopoietic stem cells. Sci Signal 2, ra75. 10.1126/scisignal.2000559.
Chen, J. Y., Miyanishi, M., Wang, S. K., Yamazaki, S., Sinha, R., Kao, K. S., Seita, J., Sahoo, D., Nakauchi, H., and Weissman, I. L. (2016). Hoxb5 marks long-term haematopoietic stem cells and reveals a homogenous perivascular niche. Nature 530, 223-227. 10.1038/nature 16943.
Chhabra, A., Ring, A. M., Weiskopf, K., Schnorr, P. J., Gordon, S., Le, A. C., Kwon, H. S., Ring, N. G., Volkmer, J., Ho, P. Y., et al. (2016). Hematopoietic stem cell transplantation in immunocompetent hosts without radiation or chemotherapy. Sci Transl Med 8, 351ra105.
Corces, M. R., Buenrostro, J. D., Wu, B., Greenside, P. G., Chan, S. M., Koenig, J. L., Snyder, M. P., Pritchard, J. K., Kundaje, A., Greenleaf, W. J., et al. (2016). Lineage-specific and single-cell chromatin accessibility charts human hematopoiesis and leukemia evolution. Nat Genet 48, 1193-1203. 10.1038/ng.3646.
Czechowicz, A., Kraft, D., Weissman, I. L., and Bhattacharya, D. (2007). Efficient transplantation via antibody-based clearance of hematopoietic stem cell niches. Science 318, 1296-1299.
Dykstra, B., Kent, D., Bowie, M., McCaffrey, L., Hamilton, M., Lyons, K., Lee, S. -J., Brinkman, R., and Eaves, C. (2007). Long-Term Propagation of Distinct Hematopoietic Differentiation Programs In Vivo. Cell Stem Cell 1, 218-229. 10.1016/j. stem.2007.05.015.
Elyahu, Y., Hekselman, I., Eizenberg-Magar, I., Berner, O., Strominger, I., Schiller, M., Mittal, K., Nemirovsky, A., Eremenko, E., Vital, A., et al. (2019). Aging promotes reorganization of the CD4 T cell landscape toward extreme regulatory and effector phenotypes. Sci Adv 5, eaaw8330.
Engblom, C., Pfirschke, C., and Pittet, M. J. (2016). The role of myeloid cells in cancer therapies. Nat Rev Cancer 16, 447-462. 10.1038/nrc.2016.54.
Flach, J., Bakker, S. T., Mohrin, M., Conroy, P. C., Pietras, E. M., Reynaud, D., Alvarez, S., Diolaiti, M. E., Ugarte, F., Forsberg, E. C., et al. (2014). Replication stress is a potent driver of functional decline in ageing haematopoietic stem cells. Nature 512, 198-202.
Florian, M. C., Leins, H., Gobs, M., Han, Y., Marka, G., Soller, K., Vollmer, A., Sakk, V., Nattamai, K. J., Rayes, A., et al. (2020). Inhibition of Cdc42 activity extends lifespan and decreases circulating inflammatory cytokines in aged female C57BL/6 mice. Aging Cell 19, e13208.
Frasca, D., Romero, M., Garcia, D., Diaz, A., and Blomberg, B. B. (2021). Hyper-metabolic B cells in the spleens of old mice make antibodies with autoimmune specificities. Immun Ageing 18, 9. 10.1186/s12979-021-00222-3.
Gekas, C., and Graf, T. (2013). CD41 expression marks myeloid-biased adult hematopoietic stem cells and increases with age. Blood 121, 4463-4472. 10.1182/blood-2012-09-457929.
George, B. M., Kao, K. S., Kwon, H. S., Velasco, B. J., Poyser, J., Chen, A., Le, A. C., Chhabra, A., Burnett, C. E., Cajuste, D., et al. (2019). Antibody Conditioning Enables MHC-Mismatched Hematopoietic Stem Cell Transplants and Organ Graft Tolerance. Cell Stem Cell 25, 185-192.e183.
Grassberger, C., Ellsworth, S. G., Wilks, M. Q., Keane, F. K., and Loeffler, J. S. (2019). Assessing the interactions between radiotherapy and antitumour immunity. Nat Rev Clin Oncol 16, 729-745. 10.1038/s41571-019-0238-9.
Guidi, N., Marka, G., Sakk, V., Zheng, Y., Florian, M.C., and Geiger, H. (2021). An aged bone marrow niche restrains rejuvenated hematopoietic stem cells. STEM CELLS 39, 1101-1106.
Gulati, G. S., Zukowska, M., Noh, J. J., Zhang, A., Wesche, D. J., Sinha, R., George, B. M., Weissman, I. L., and Szade, K. (2019). Neogenin-1 distinguishes between myeloid-biased and balanced. Proc Natl Acad Sci USA 116, 25115-25125.
Guo, G., Luc, S., Marco, E., Lin, T. -W., Peng, C., Marc, Beyaz, S., Kim, W., Xu, J., Partha, et al. (2013). Mapping Cellular Hierarchy by Single-Cell Analysis of the Cell Surface Repertoire. Cell Stem Cell 13, 492-505. 10.1016/j.stem.2013.07.017.
Hao, Y., O'Neill, P., Naradikian, M. S., Scholz, J. L., and Cancro, M. P. (2011). A B-cell subset uniquely responsive to innate stimuli accumulates in aged mice. Blood 118, 1294-1304.
Hennrich, M. L., Romanov, N., Horn, P., Jaeger, S., Eckstein, V., Steeples, V., Ye, F., Ding, X., Poisa-Beiro, L., Lai, M. C., et al. (2018). Cell-specific proteome analyses of human bone marrow reveal molecular features of age-dependent functional decline. Nat Commun 9, 4004.
Hu, Z., Ott, P. A., and Wu, C. J. (2018). Towards personalized, tumour-specific, therapeutic vaccines for cancer. Nat Rev Immunol 18, 168-182. 10.1038/nri.2017.131.
Jaiswal, S., and Ebert, B. L. (2019). Clonal hematopoiesis in human aging and disease. Science 366. 10.1126/science.aan4673.
Jaiswal, S., Jamieson, C. H., Pang, W. W., Park, C. Y., Chao, M. P., Majeti, R., Traver, D., van Rooijen, N., and Weissman, I. L. (2009). CD47 is upregulated on circulating hematopoietic stem cells and leukemia cells to avoid phagocytosis. Cell 138, 271-285. 10.1016/j.cell.2009.05.046.
Jaiswal, S., and Weissman, I. L. (2009). Hematopoietic stem and progenitor cells and the inflammatory response. Ann NY Acad Sci 1174, 118-121. 10.1111/j.1749-6632.2009.04930.x.
Karsunky, H., Inlay, M. A., Serwold, T., Bhattacharya, D., and Weissman, I. L. (2008). Flk2+ common lymphoid progenitors possess equivalent differentiation potential for the B and T lineages. Blood 111, 5562-5570. 10.1182/blood-2007-11-126219.
Keren, Z., Naor, S., Nussbaum, S., Golan, K., Itkin, T., Sasaki, Y., Schmidt-Supprian, M., Lapidot, T., and Melamed, D. (2011). B-cell depletion reactivates B lymphopoiesis in the BM and rejuvenates the B lineage in aging. Blood 117, 3104-3112. 10.1182/blood-2010-09-307983.
Kondo, M., Weissman, I. L., and Akashi, K. (1997). Identification of clonogenic common lymphoid progenitors in mouse bone marrow. Cell 91, 661-672. 10.1016/s0092-8674(00)80453-5.
Kumar, P., Beck, D., Galeev, R., Thoms, J. A. I., Talkhoncheh, M. S., de Jong, I., Unnikrishnan, A., Baudet, A., Subramaniam, A., Pimanda, J. E., and Larsson, J. (2019). HMGA2 promotes long-term engraftment and myeloerythroid differentiation of human hematopoietic stem and progenitor cells. Blood Adv 3, 681-691. 10.1182/bloodadvances.2018023986.
Leins, H., Mulaw, M., Eiwen, K., Sakk, V., Liang, Y., Denkinger, M., Geiger, H., and Schirmbeck, R. (2018). Aged murine hematopoietic stem cells drive aging-associated immune remodeling. Blood 132, 565-576. 10.1182/blood-2018-02-831065.
Li, F., and Ravetch, J. V. (2012). A general requirement for FcyRIIB co-engagement of agonistic anti-TNFR antibodies. Cell Cycle 11, 3343-3344. 10.4161/cc.21842.
Loder, F., Mutschler, B., Ray, R. J., Paige, C. J., Sideras, P., Torres, R., Lamers, M. C., and Carsetti, R. (1999). B cell development in the spleen takes place in discrete steps and is determined by the quality of B cell receptor-derived signals. J Exp Med 190, 75-89.
Luckey, C. J., Bhattacharya, D., Goldrath, A. W., Weissman, I. L., Benoist, C., and Mathis, D. (2006). Memory T and memory B cells share a transcriptional program of self-renewal with long-term hematopoietic stem cells. Proc Natl Acad Sci USA 103, 3304-3309.
Mahi, N. A., Najafabadi, M. F., Pilarczyk, M., Kouril, M., and Medvedovic, M. (2019). GREIN: An Interactive Web Platform for Re-analyzing GEO RNA-seq Data. Sci Rep 9, 7580.
Majeti, R. (2014). Clonal evolution of pre-leukemic hematopoietic stem cells precedes human acute myeloid leukemia. Best Pract Res Clin Haematol 27, 229-234.
Mann, M., Mehta, A., De Boer, C. G., Kowalczyk, M. S., Lee, K., Haldeman, P., Rogel, N., Knecht, A. R., Farouq, D., Regev, A., and Baltimore, D. (2018). Heterogeneous Responses of Hematopoietic Stem Cells to Inflammatory Stimuli Are Altered with Age. Cell Reports 25, 2992-3005.e2995. 10.1016/j.celrep.2018.11.056.
Maryanovich, M., Zahalka, A. H., Pierce, H., Pinho, S., Nakahara, F., Asada, N., Wei, Q., Wang, X., Ciero, P., Xu, J., et al. (2018). Adrenergic nerve degeneration in bone marrow drives aging of the hematopoietic stem cell niche. Nat Med 24, 782-791. 10.1038/s41591-018-0030-X.
Montecino-Rodriguez, E., Kong, Y., Casero, D., Rouault, A., Dorshkind, K., and Pioli, P. D. (2019). Lymphoid-Biased Hematopoietic Stem Cells Are Maintained with Age and Efficiently Generate Lymphoid Progeny. Stem Cell Reports 12, 584-596. 10.1016/j.stemcr.2019.01.016.
Morrison, S. J., Wandycz, A. M., Akashi, K., Globerson, A., and Weissman, I. L. (1996). The aging of hematopoietic stem cells. Nat Med 2, 1011-1016. 10.1038/nm0996-1011.
Nikolich-Žugich, J. (2018). The twilight of immunity: emerging concepts in aging of the immune system. Nat Immunol 19, 10-19. 10.1038/s41590-017-0006-x.
Nimmerjahn, F., and Ravetch, J. V. (2005). Divergent immunoglobulin g subclass activity through selective Fc receptor binding. Science 310, 1510-1512. 10.1126/science.1118948.
Nimmerjahn, F., and Ravetch, J. V. (2006). Fcgamma receptors: old friends and new family members. Immunity 24, 19-28. 10.1016/j.immuni.2005.11.010.
Nimmerjahn, F., and Ravetch, J. V. (2008). Fcgamma receptors as regulators of immune responses. Nat Rev Immunol 8, 34-47. 10.1038/nri2206.
Norddahl, G. L., Pronk, C. J., Wahlestedt, M., Sten, G., Nygren, J. M., Ugale, A., Sigvardsson, M., and Bryder, D. (2011). Accumulating mitochondrial DNA mutations drive premature hematopoietic aging phenotypes distinct from physiological stem cell aging. Cell Stem Cell 8, 499-510. 10.1016/j.stem.2011.03.009.
Palmer, S., Albergante, L., Blackburn, C. C., and Newman, T. J. (2018). Thymic involution and rising disease incidence with age. Proc Natl Acad Sci USA 115, 1883-1888.
Pang, W. W., Pluvinage, J. V., Price, E. A., Sridhar, K., Arber, D. A., Greenberg, P. L., Schrier, S. L., Park, C. Y., and Weissman, I. L. (2013). Hematopoietic stem cell and progenitor cell mechanisms in myelodysplastic syndromes. Proc Natl Acad Sci USA 110, 3011-3016.
Pang, W. W., Price, E. A., Sahoo, D., Beerman, I., Maloney, W. J., Rossi, D. J., Schrier, S. L., and Weissman, I. L. (2011). Human bone marrow hematopoietic stem cells are increased in frequency and myeloid-biased with age. Proc Natl Acad Sci USA 108, 20012-20017.
Pang, W. W., Schrier, S. L., and Weissman, I. L. (2017). Age-associated changes in human hematopoietic stem cells. Semin Hematol 54 39-42. 10.1053/j.seminhematol.2016.10.004.
Park, C. Y., Majeti, R., and Weissman, I. L. (2008). In vivo evaluation of human hematopoiesis through xenotransplantation of purified hematopoietic stem cells from umbilical cord blood. Nat Protoc 3, 1932-1940. 10.1038/nprot.2008.194.
Péault, B., Weissman, I. L., Buckle, A. M., Tsukamoto, A., and Baum, C. (1993). Thy-1-expressing CD34+ human cells express multiple hematopoietic potentialities in vitro and in SCID-hu mice. Nouv Rev Fr Hematol 35, 91-93.
Pronk, C. J. H., Rossi, D. J., Månsson, R., Attema, J. L., Norddahl, G. L., Chan, C. K. F., Sigvardsson, M., Weissman, I. L., and Bryder, D. (2007). Elucidation of the Phenotypic, Functional, and Molecular Topography of a Myeloerythroid Progenitor Cell Hierarchy. Cell Stem Cell 1, 428-442. 10.1016/j.stem.2007.07.005.
Ratliff, M., Alter, S., Frasca, D., Blomberg, B. B., and Riley, R. L. (2013). In senescence, age-associated B cells secrete TNFa and inhibit survival of B-cell precursors. Aging Cell 12, 303-311.
Renders, S., Svendsen, A. F., Panten, J., Rama, N., Maryanovich, M., Sommerkamp, P., Ladel, L., Redavid, A. R., Gibert, B., Lazare, S., et al. (2021). Niche derived netrin-1 regulates hematopoietic stem cell dormancy via its receptor neogenin-1. Nat Commun 12, 608.
Rossi, D. J., Bryder, D., and Weissman, I. L. (2007). Hematopoietic stem cell aging: mechanism and consequence. Exp Gerontol 42, 385-390. 10.1016/j.exger.2006.11.019.
Rossi, D. J., Jamieson, C. H., and Weissman, I. L. (2008). Stems cells and the pathways to aging and cancer. Cell 132, 681-696. 10.1016/j.cell.2008.01.036.
Rundberg Nilsson, A., Soneji, S., Adolfsson, S., Bryder, D., and Pronk, C. J. (2016). Human and Murine Hematopoietic Stem Cell Aging Is Associated with Functional Impairments and Intrinsic Megakaryocytic/Erythroid Bias. PLOS One 11, e0158369.
Sanjuan-Pla, A., Macaulay, I. C., Jensen, C. T., Woll, P. S., Luis, T. C., Mead, A., Moore, S., Carella, C., Matsuoka, S., Bouriez Jones, T., et al. (2013). Platelet-biased stem cells reside at the apex of the haematopoietic stem-cell hierarchy. Nature 502, 232-236. 10.1038/nature12495.
Säwén, P., Lang, S., Mandal, P., Rossi, D. J., Soneji, S., and Bryder, D. (2016). Mitotic History Reveals Distinct Stem Cell Populations and Their Contributions to Hematopoiesis. Cell Rep 14, 2809-2818. 10.1016/j.celrep.2016.02.073.
Seita, J., Sahoo, D., Rossi, D. J., Bhattacharya, D., Serwold, T., Inlay, M. A., Ehrlich, L. I., Fathman, J. W., Dill, D. L., and Weissman, I. L. (2012). Gene Expression Commons: an open platform for absolute gene expression profiling. PLOS One 7, e40321.
Seita, J., and Weissman, I. L. (2010). Hematopoietic stem cell: self-renewal versus differentiation. Wiley Interdiscip Rev Syst Biol Med 2, 640-653. 10.1002/wsbm.86.
Siegel, R. L., Miller, K. D., Fuchs, H. E., and Jemal, A. (2022). Cancer statistics, 2022. CA Cancer J Clin 72, 7-33. 10.3322/caac.21708.
Spangrude, G. J., Heimfeld, S., and Weissman, I. L. (1988). Purification and characterization of mouse hematopoietic stem cells. Science 241, 58-62. 10.1126/science.2898810.
Sudo, T., Nishikawa, S., Ohno, N., Akiyama, N., Tamakoshi, M., and Yoshida, H. (1993). Expression and function of the interleukin 7 receptor in murine lymphocytes. Proc Natl Acad Sci USA 90, 9125-9129. 10.1073/pnas.90.19.9125.
Sun, et al. (2014). Epigenomic profiling of young and aged HSCs reveals concerted changes during aging that reinforce self-renewal. Cell Stem Cell 14, 673-688.
Thomas, R., Wang, W., and Su, D. M. (2020). Contributions of Age-Related Thymic Involution to Immunosenescence and Inflammaging. Immun Ageing 17, 2. 10.1186/s12979-020-0173-8.
Tong, J., Sun, T., Ma, S., Zhao, Y., Ju, M., Gao, Y., Zhu, P., Tan, P., Fu, R., Zhang, A., et al. (2021). Hematopoietic stem cell heterogeneity is linked to the initiation and therapeutic response of myeloproliferative neoplasms. Cell Stem Cell 28, 780. 10.1016/j.stem.2021.02.026.
Uchida, N., Tsukamoto, A., He, D., Friera, A. M., Scollay, R., and Weissman, I. L. (1998). High doses of purified stem cells cause early hematopoietic recovery in syngeneic and allogeneic hosts. J Clin Invest 101, 961-966. 10.1172/JCI1681.
Valletta, S., Thomas, A., Meng, Y., Ren, X., Drissen, R., Sengül, H., Di Genua, C., and Nerlov, C. (2020). Micro-environmental sensing by bone marrow stroma identifies IL-6 and TGFβ1 as regulators of hematopoietic ageing. Nat Commun 11, 4075. 10.1038/s41467-020-17942-7.
Wahlestedt, M., Norddahl, G. L., Sten, G., Ugale, A., Frisk, M. A., Mattsson, R., Deierborg, T., Sigvardsson, M., and Bryder, D. (2013). An epigenetic component of hematopoietic stem cell aging amenable to reprogramming into a young state. Blood 121, 4257-4264. 10.1182/blood-2012-11-469080.
Weiner, L. M., Surana, R., and Wang, S. (2010). Monoclonal antibodies: versatile platforms for cancer immunotherapy. Nat Rev Immunol 10, 317-327. 10.1038/nri2744.
Weiskopf, K., Schnorr, P. J., Pang, W. W., Chao, M. P., Chhabra, A., Seita, J., Feng, M., and Weissman, I. L. (2016). Myeloid Cell Origins, Differentiation, and Clinical Implications. Microbiol Spectr 4. 10.1128/microbiolspec. MCHD-0031-2016.
Weissman, I. L. (2000). Stem cells: units of development, units of regeneration, and units in evolution. Cell 100, 157-168. 10.1016/s0092-8674(00)81692-x.
Weissman, I. L. (2015a). Stem cells are units of natural selection for tissue formation, for germline development, and in cancer development. Proceedings of the National Academy of Sciences 112, 8922-8928. 10.1073/pnas.1505464112.
Weissman, I. L. (2015b). Stem cells are units of natural selection for tissue formation, for germline development, and in cancer development. Proc Natl Acad Sci USA 112, 8922-8928. 10.1073/pnas.1505464112.
Woll, P. S., Kjällquist, U., Chowdhury, O., Doolittle, H., Wedge, D. C., Thongjuea, S., Erlandsson, R., Ngara, M., Anderson, K., Deng, Q., et al. (2014). Myelodysplastic syndromes are propagated by rare and distinct human cancer stem cells in vivo. Cancer Cell 25, 794-808.
Wu, W. C., Sun, H. W., Chen, H. T., Liang, J., Yu, X. J., Wu, C., Wang, Z., and Zheng, L. (2014). Circulating hematopoietic stem and progenitor cells are myeloid-biased in cancer patients. Proc Natl Acad Sci USA 111, 4221-4226. 10.1073/pnas.1320753111.
Yamamoto, R., Morita, Y., Ooehara, J., Hamanaka, S., Onodera, M., Rudolph, K. L., Ema, H., and Nakauchi, H. (2013). Clonal analysis unveils self-renewing lineage-restricted progenitors generated directly from hematopoietic stem cells. Cell 154, 1112-1126.
Yamamoto, R., Wilkinson, A. C., Ooehara, J., Lan, X., Lai, C. -Y., Nakauchi, Y., Pritchard, J. K., and Nakauchi, H. (2018). Large-Scale Clonal Analysis Resolves Aging of the Mouse Hematopoietic Stem Cell Compartment. Cell Stem Cell 22, 600-607.e604.
Zaro, B. W., Noh, J. J., Mascetti, V. L., Demeter, J., George, B., Zukowska, M., Gulati, G. S., Sinha, R., Flynn, R. A., Banuelos, A., et al. (2020). Proteomic analysis of young and old mouse hematopoietic stem cells and their progenitors reveals post-transcriptional regulation in stem cells. Elife 9. 10.7554/eLife.62210.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

1. A method of rebalancing the immune system of a mammalian subject by selective depletion of myeloid-biased hematopoietic stem cells (my-HSC) relative to balanced hematopoietic stem cells (bal-HSC), the method comprising:
administering to the subject one or a cocktail of agents specific for a cell surface marker differentially expressed on my-HSC relative to bal-HSC; in a dose effective to selectively deplete the my-HSC.

2. The method of claim 1, wherein the cell surface marker differentially expressed on my-HSC relative to bal-HSC is selected from CD150 (Slamf1), CD61 (Itgb3), CD41 (Itga2b), CD62p, and NEO1.

3. The method of claim 1,wherein the cell surface marker differentially expressed on my-HSC relative to bal-HSC is selected from CD150 (Slamf1), CD62p, and NEO1.

4. The method of claim 1, wherein the agent specific for a cell surface marker differentially expressed on my-HSC relative to bal-HSC is an antibody specific for the cell surface marker.

5. The method of claim 1, further comprising administering to the subject an agent that blocks CD47 interaction with SIRPE.

6. The method of claim 5, wherein administration of subject an agent that blocks CD47 interaction with SIRPE is performed concomitantly with administration of one or a cocktail of agents specific for a cell surface marker differentially expressed on my-HSC relative to bal-HSC.

7. The method of claim 5, wherein the agent that blocks CD47 interaction with SIRP [symbol] is selected from an anti-CD47 antibody, an anti-SIRPα antibody, and a soluble SIRPα polypeptide.

8. The method of claim 1, further comprising administering to the subject an agent that specifically binds to CD117.

9. The method of claim 8, wherein the agent that specifically binds to CD117 is an antibody.

10. The method of claim 8 wherein administration of subject an agent that blocks specifically binds to CD117 is performed concomitantly with administration of one or a cocktail of agents specific for a cell surface marker differentially expressed on my-HSC relative to bal-HSC.

11. The method of claim 1, comprising administering a combination of an agent that blocks CD47 interaction with SIRP [symbol] , an antibody specific for CD117, and an antibody specific for CD62p.

12. The method of claim 1, comprising administering a combination of an agent that blocks CD47 interaction with SIRP [symbol] , an antibody specific for CD117, and an antibody specific for CD150.

13. The method of claim 1, comprising administering a combination of an agent that blocks CD47 interaction with SIRP [symbol] , an antibody specific for CD117, and an antibody specific for NEO1.

14. The method of claim 1, wherein the subject is an elderly human.

15. The method of claim 1, wherein following the administering step, there is an enrichment of bal-HSC to my-HSC of from at least 1.5-fold to at least 15-fold.

16. The method of claim 1, wherein following the administering step, the ratio of the number of lymphoid progenitors in bone marrow of the subject to the number of myeloid progenitors in bone marrow of the subject is increased at least 1.5-fold fold to at least 15-fold.

17. The method of claim 1, wherein following the administering step the number of circulating naïve T cells relative to the total circulating lymphocyte population is increased at least 1.5-fold to at least 15-fold.

18. The method of claim 1, wherein the balance of immune cells including one or more of: the relative number of one or more of naïve T cells, exhausted T cells, ABC, myeloid progenitors and lymphoid progenitors, is determined before my-HSC-selective depletion.

19. The method of claim 1, wherein following the administering step, the basal circulating level of an inflammaging marker selected from: one or more of IL-1a, CXCL5, IL1RL1, IL-23, IL-1b, CXCL2, IL-31, IL-5, GM-CSF, is decreased at least 2-fold.

20. The method of claim 1, wherein following the administering step, an antigen-specific CD8+ T cell response is increased at least 1.5-fold.

21. The method of claim 1, wherein following the administering step, an antigen-specific antibody response is increased at least 1.5-fold.
